SEQUENCE LISTING (51 sequences)

SEQ ID NO: 1 - protein, 20 aa, Homo sapiens
ESTFEKLRLPSRKVDALDHF

SEQ ID NO: 2 - protein, 7 aa, Artificial Sequence
  SYNTHESIZED
  misc_feature (2)..(2): Xaa can be any naturally occurring amino acid
  MISC_FEATURE (3)..(3): Xaa is Ser or Thr
  misc_feature (4)..(4): Xaa can be any naturally occurring amino acid
  misc_feature (6)..(7): Xaa can be any naturally occurring amino acid
LXXXVXX

SEQ ID NO: 3 - protein, 19 aa, Artificial Sequence
  SYNTHESIZED
  MISC_FEATURE (1)..(1): Xaa is Ser or Thr
  misc_feature (2)..(2): Xaa can be any naturally occurring amino acid
  MISC_FEATURE (3)..(3): Xaa is Ser or Thr
  MISC_FEATURE (6)..(6): Xaa is Phe or Tyr
  MISC_FEATURE (7)..(8): Xaa is Arg or Lys
  MISC_FEATURE (10)..(10): Xaa is Ser or Thr
  misc_feature (11)..(12): Xaa can be any naturally occurring amino acid
  MISC_FEATURE (18)..(18): Xaa is Phe or Tyr
XXXGGXXXGXXXGHDVDXL

SEQ ID NO: 4 - protein, 5 aa, Artificial Sequence
  SYNTHESIZED
  misc_feature (2)..(3): Xaa can be any naturally occurring amino acid
  MISC_FEATURE (4)..(4): Xaa is Phe or Tyr
  misc_feature (5)..(5): Xaa can be any naturally occurring amino acid
QXXXX

SEQ ID NO: 5 - protein, 6 aa, Artificial Sequence
  SYNTHESIZED
  misc_feature (1)..(1): Xaa can be any naturally occurring amino acid
  MISC_FEATURE (2)..(2): Xaa is Arg or Lys
XXVDLV

SEQ ID NO: 6 - protein, 23 aa, Artificial Sequence
  SYNTHESIZED
  misc_feature (1)..(1): Xaa can be any naturally occurring amino acid
  MISC_FEATURE (2)..(2): Xaa is Phe or Tyr
  MISC_FEATURE (8)..(8): Xaa is Ser or Thr
  MISC_FEATURE (10)..(10): Xaa is Ser or Thr
  MISC_FEATURE (11)..(11): Xaa is Arg or Lys
  misc_feature (12)..(12): Xaa can be any naturally occurring amino acid
  MISC_FEATURE (13)..(13): Xaa is Phe or Tyr
  misc_feature (14)..(14): Xaa can be any naturally occurring amino acid
  MISC_FEATURE (15)..(15): Xaa is Arg or Lys
  misc_feature (16)..(16): Xaa can be any naturally occurring amino acid
  MISC_FEATURE (18)..(19): Xaa is Arg or Lys
  MISC_FEATURE (20)..(20): Xaa is Phe or Tyr
  misc_feature (21)..(23): Xaa can be any naturally occurring amino acid
XXALLGWXGXXXXXXXLXXXXXX

SEQ ID NO: 7 - protein, 6 aa, Artificial Sequence
  SYNTHESIZED
  misc_feature (1)..(3): Xaa can be any naturally occurring amino acid
  misc_feature (5)..(6): Xaa can be any naturally occurring amino acid
XXXLXX

SEQ ID NO: 8 - protein, 520 aa, Bos taurus
MAQQRQHQRLPMDPLCTASSGPRKKRPRQVGASMASPPHDIKFQNLVLFILEKKMGTTRRNFLMELARRKGFRVENELSDSVTHIVAENNSGSEVLEWLQVQNIRASSQLELLDVSWLIESMGAGKPVEITGKHQLVVRTDYSATPNPGFQKTPPLAVKKISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 9 - protein, 383 aa, Bos taurus
MRTDYSATPNPGFQKTPPLAVKKISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 10 - protein, 510 aa, Mus musculus
MDPLQAVHLGPRKKRPRQLGTPVASTPYDIRFRDLVLFILEKKMGTTRRAFLMELARRKGFRVENELSDSVTHIVAENNSGSDVLEWLQLQNIKASSELELLDISWLIECMGAGKPVEMMGRHQLVVNRNSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 11 - protein, 384 aa, Mus musculus
MNRNSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 12 - protein, 517 aa, Sarciphilus harrisii
MHRIRTTDSDHGKKRQKKMDAISSKLYEIKFHEFVLFILEKKMGATRRTFLMDLARKKGFRVESELSNSVTHIVAENNSGSDVLAWLEAHKLETTAHFELLDVSWLIECMKVGKPVDTKGKYQLVESSIASANPDPNEGMLKIQSPAMNAISPYACQRRTTLNNHNQRFTDAFEILAKNYEFRENHGHCLTFLRATSVLKCLPFAIVSMKDAEGLPWIGDEVKGIMEEIIEDGQSLEVQAVLNDERYQAFKLFTSVFGVGLKTAEKWYRMGFRTLSKIQSDKSLKFTKMQKAGFLYYEDLISCVSKAEADAVSLIVKEAVWTFLPDALITITGGFRRGKEFGHDVDFLITSPGGEKEQVDQLLQKVTNLWEKQGLLLYYDLMESTFEDLKLPSRKVDALDHFQKCFLILKLYCQRGDRSKWEGPEGSNGLQTKNWKAIRVDLVVCPYDRYAYALLGWSGSRQFERDLRRYATHEKKMMLDNHALYDKTKRTFLKAESEEEIFSHLGLEYIEPWERNA

SEQ ID NO: 13 - protein, 390 aa, Sarciphilus harrisii
MIASANPDPNEGMLKIQSPAMNAISPYACQRRTTLNNHNQRFTDAFEILAKNYEFRENHGHCLTFLRATSVLKCLPFAIVSMKDAEGLPWIGDEVKGIMEEIIEDGQSLEVQAVLNDERYQAFKLFTSVFGVGLKTAEKWYRMGFRTLSKIQSDKSLKFTKMQKAGFLYYEDLISCVSKAEADAVSLIVKEAVWTFLPDALITITGGFRRGKEFGHDVDFLITSPGGEKEQVDQLLQKVTNLWEKQGLLLYYDLMESTFEDLKLPSRKVDALDHFQKCFLILKLYCQRGDRSKWEGPEGSNGLQTKNWKAIRVDLVVCPYDRYAYALLGWSGSRQFERDLRRYATHEKKMMLDNHALYDKTKRTFLKAESEEEIFSHLGLEYIEPWERNA

SEQ ID NO: 14 - protein, 494 aa, Homo sapiens
MLPKRRRARVGSPSGDAASSTPPSTRFPGVAIYLVEPRMGRSRRAFLTGLARSKGFRVLDACSSEATHVVMEETSAEEAVSWQERRMAAAPPGCTPPALLDISWLTESLGAGQPVPVECRHRLEVAGPRKGPLSPAWMPAYACQRPTPLTHHNTGLSEALEILAEAAGFEGSEGRLLTFCRAASVLKALPSPVTTLSQLQGLPHFGEHSSRVVQELLEHGVCEEVERVRRSERYQTMKLFTQIFGVGVKTADRWYREGLRTLDDLREQPQKLTQQQKAGLQHHQDLSTPVLRSDVDALQQVVEEAVGQALPGATVTLTGGFRRGKLQGHDVDFLITHPKEGQEAGLLPRVMCRLQDQGLILYHQHQHSCCESPTRLAQQSHMDAFERSFCIFRLPQPPGAAVGGSTRPCPSWKAVRVDLVVAPVSQFPFALLGWTGSKLFQRELRRFSRKEKGLWLNSHGLFDPEQKTFFQAASEEDIFRHLGLEYLPPEQRNA

SEQ ID NO: 15 - protein, 495 aa, Artificial Sequence
  SYNTHESIZED
MLPKRRRARVGSPSGDAASSTPPSTRFPGVAIYLVEPRMGRSRRAFLTGLARSKGFRVLDACSSEATHVVMEETSAEEAVSWQERRMAAAPPGCTPPALLDISWLTESLGAGQPVPVECRHRLEVAGPRKGPLSPAWMPAYACQRPTPLTHHNTGLSEALEILAEAAGFEGSEGRLLTFCRAASVLKALPSPVTTLSQLQGLPHFGEHSSRVVQELLEHGVCEEVERVRRSERYQTMKLFTQIFGVGVKTADRWYREGLRTLDDLREQPQKLTQQQKAGLQHHQDLSTPVLRSDVDALQQVVEEAVGQALPGATVTLTGGFRRGKLQGHDVDFLITHPKEGQEAGLLPRVMCRLQDQGLILYHQHQESTFEKLRLPSRKVDALDHFERSFCIFRLPQPPGAAVGGSTRPCPSWKAVRVDLVVAPVSQFPFALLGWTGSKLFQRELRRFSRKEKGLWLNSHGLFDPEQKTFFQAASEEDIFRHLGLEYLPPEQRNA

SEQ ID NO: 16 - protein, 388 aa, Artificial Sequence
  SYNTHESIZED
MLGAGQPVPVECRHRLEVAGPRKGPLSPAWMPAYACQRPTPLTHHNTGLSEALEILAEAAGFEGSEGRLLTFCRAASVLKALPSPVTTLSQLQGLPHFGEHSSRVVQELLEHGVCEEVERVRRSERYQTMKLFTQIFGVGVKTADRWYREGLRTLDDLREQPQKLTQQQKAGLQHHQDLSTPVLRSDVDALQQVVEEAVGQALPGATVTLTGGFRRGKLQGHDVDFLITHPKEGQEAGLLPRVMCRLQDQGLILYHQHQESTFEKLRLPSRKVDALDHFERSFCIFRLPQPPGAAVGGSTRPCPSWKAVRVDLVVAPVSQFPFALLGWTGSKLFQRELRRFSRKEKGLWLNSHGLFDPEQKTFFQAASEEDIFRHLGLEYLPPEQRNA

SEQ ID NO: 17 - protein, 575 aa, Homo sapiens
MDPRGILKAFPKRQKIHADASSKVLAKIPRREEGEEAEEWLSSLRAHVVRTGIGRARAELFEKQIVQHGGQLCPAQGPGVTHIVVDEGMDYERALRLLRLPQLPPGAQLVKSAWLSLCLQERRLVDVAGFSIFIPSRYLDHPQPSKAEQDASIPPGTHEALLQTALSPPPPPTRPVSPPQKAKEAPNTQAQPISDDEASDGEETQVSAADLEALISGHYPTSLEGDCEPSPAPAVLDKWVCAQPSSQKATNHNLHITEKLEVLAKAYSVQGDKWRALGYAKAINALKSFHKPVTSYQEACSIPGIGKRMAEKIIEILESGHLRKLDHISESVPVLELFSNIWGAGTKTAQMWYQQGFRSLEDIRSQASLTTQQAIGLKHYSDFLERMPREEATEIEQTVQKAAQAFNSGLLCVACGSYRRGKATCGDVDVLITHPDGRSHRGIFSRLLDSLRQEGFLTDDLVSQEENGQQQKYLGVCRLPGPGRRHRRLDIIVVPYSEFACALLYFTGSAHFNRSMRALAKTKGMSLSEHALSTAVVRNTHGCKVGPGRVLPTPTEKDVFRLLGLPYREPAERDW

SEQ ID NO: 18 - protein, 587 aa, Artificial Sequence
  SYNTHESIZED
MDPRGILKAFPKRQKIHADASSKVLAKIPRREEGEEAEEWLSSLRAHVVRTGIGRARAELFEKQIVQHGGQLCPAQGPGVTHIVVDEGMDYERALRLLRLPQLPPGAQLVKSAWLSLCLQERRLVDVAGFSIFIPSRYLDHPQPSKAEQDASIPPGTHEALLQTALSPPPPPTRPVSPPQKAKEAPNTQAQPISDDEASDGEETQVSAADLEALISGHYPTSLEGDCEPSPAPAVLDKWVCAQPSSQKATNHNLHITEKLEVLAKAYSVQGDKWRALGYAKAINALKSFHKPVTSYQEACSIPGIGKRMAEKIIEILESGHLRKLDHISESVPVLELFSNIWGAGTKTAQMWYQQGFRSLEDIRSQASLTTQQAIGLKHYSDFLERMPREEATEIEQTVQKAAQAFNSGLLCVACGSYRRGKATCGDVDVLITHPDGRSHRGIFSRLLDSLRQEGFLTDDLVESTFEKLRLPSRKVDALDHFQKYLGVCRLPGPGRRHRRLDIIVVPYSEFACALLYFTGSAHFNRSMRALAKTKGMSLSEHALSTAVVRNTHGCKVGPGRVLPTPTEKDVFRLLGLPYREPAERDW

SEQ ID NO: 19 - protein, 383 aa, Artificial Sequence
  SYNTHESIZED
MVSAADLEALISGHYPTSLEGDCEPSPAPAVLDKWVCAQPSSQKATNHNLHITEKLEVLAKAYSVQGDKWRALGYAKAINALKSFHKPVTSYQEACSIPGIGKRMAEKIIEILESGHLRKLDHISESVPVLELFSNIWGAGTKTAQMWYQQGFRSLEDIRSQASLTTQQAIGLKHYSDFLERMPREEATEIEQTVQKAAQAFNSGLLCVACGSYRRGKATCGDVDVLITHPDGRSHRGIFSRLLDSLRQEGFLTDDLVESTFEKLRLPSRKVDALDHFQKYLGVCRLPGPGRRHRRLDIIVVPYSEFACALLYFTGSAHFNRSMRALAKTKGMSLSEHALSTAVVRNTHGCKVGPGRVLPTPTEKDVFRLLGLPYREPAERDW

SEQ ID NO: 20 - protein, 335 aa, Homo sapiens
MSKRKAPQETLNGGITDMLTELANFEKNVSQAIHKYNAYRKAASVIAKYPHKIKSGAEAKKLPGVGTKIAEKIDEFLATGKLRKLEKIRQDDTSSSINFLTRVSGIGPSAARKFVDEGIKTLEDLRKNEDKLNHHQRIGLKYFGDFEKRIPREEMLQMQDIVLNEVKKVDSEYIATVCGSFRRGAESSGDMDVLLTHPSFTSESTKQPKLLHQVVEQLQKVHFITDTLSKGETKFMGVCQLPSKNDEKEYPHRRIDIRLIPKDQYYCGVLYFTGSDIFNKNMRAHALEKGFTINEYTIRPLGVTGVAGEPLPVDSEKDIFDYIQWKYREPKDRSE

SEQ ID NO: 21 - protein, 352 aa, Artificial Sequence
  SYNTHESIZED
MSKRKAPQETLNGGITDMLTELANFEKNVSQAIHKYNAYRKAASVIAKYPHKIKSGAEAKKLPGVGTKIAEKIDEFLATGKLRKLEKIRQDDTSSSINFLTRVSGIGPSAARKFVDEGIKTLEDLRKNEDKLNHHQRIGLKYFGDFEKRIPREEMLQMQDIVLNEVKKVDSEYIATVCGSFRRGAESSGDMDVLLTHPSFTSESTKQPKLLHQVVEQLQKVHFITDTLSKESTFEKLRLPSRKVDALDHFKFMGVCQLPSKNDEKEYPHRRIDIRLIPKDQYYCGVLYFTGSDIFNKNMRAHALEKGFTINEYTIRPLGVTGVAGEPLPVDSEKDIFDYIQWKYREPKDRSE

SEQ ID NO: 22 - protein, 174 aa, African swine fever virus
MLTLIQGKKIVNHLRSRLAFEYNGQLIKILSKNIVAVGSLRREEKMLNDVDLLIIVPEKKLLKHVLPNIRIKGLSFSVKVCGERKCVLFIEWEKKTYQLDLFTALAEEKPYAIFHFTGPVSYLIRIRAALKKKNYKLNQYGLFKNQTLVPLKITTEKELIKELGFTYRIPKKRL

SEQ ID NO: 23 - protein, 191 aa, Artificial Sequence
  SYNTHESIZED
MLTLIQGKKIVNHLRSRLAFEYNGQLIKILSKNIVAVGSLRREEKMLNDVDLLIIVPEKKLLKHVLPNIRIKGLSFSVKVCESTFEKLRLPSRKVDALDHFKCVLFIEWEKKTYQLDLFTALAEEKPYAIFHFTGPVSYLIRIRAALKKKNYKLNQYGLFKNQTLVPLKITTEKELIKELGFTYRIPKKRL

SEQ ID NO: 24 - protein, 2549 aa, Homo sapiens
MNLLRRSGKRRRSESGSDSFSGSGGDSSASPQFLSGSVLSPPPGLGRCLKAAAAGECKPTVPDYERDKLLLANWGLPKAVLEKYHSFGVKKMFEWQAECLLLGQVLEGKNLVYSAPTSAGKTLVAELLILKRVLEMRKKALFILPFVSVAKEKKYYLQSLFQEVGIKVDGYMGSTSPSRHFSSLDIAVCTIERANGLINRLIEENKMDLLGMVVVDELHMLGDSHRGYLLELLLTKICYITRKSASCQADLASSLSNAVQIVGMSATLPNLELVASWLNAELYHTDFRPVPLLESVKVGNSIYDSSMKLVREFEPMLQVKGDEDHVVSLCYETICDNHSVLLFCPSKKWCEKLADIIAREFYNLHHQAEGLVKPSECPPVILEQKELLEVMDQLRRLPSGLDSVLQKTVPWGVAFHHAGLTFEERDIIEGAFRQGLIRVLAATSTLSSGVNLPARRVIIRTPIFGGRPLDILTYKQMVGRAGRKGVDTVGESILICKNSEKSKGIALLQGSLKPVRSCLQRREGEEVTGSMIRAILEIIVGGVASTSQDMHTYAACTFLAASMKEGKQGIQRNQESVQLGAIEACVMWLLENEFIQSTEASDGTEGKVYHPTHLGSATLSSSLSPADTLDIFADLQRAMKGFVLENDLHILYLVTPMFEDWTTIDWYRFFCLWEKLPTSMKRVAELVGVEEGFLARCVKGKVVARTERQHRQMAIHKRFFTSLVLLDLISEVPLREINQKYGCNRGQIQSLQQSAAVYAGMITVFSNRLGWHNMELLLSQFQKRLTFGIQRELCDLVRVSLLNAQRARVLYASGFHTVADLARANIVEVEVILKNAVPFKSARKAVDEEEEAVEERRNMRTIWVTGRKGLTEREAAALIVEEARMILQQDLVEMGVQWNPCALLHSSTCSLTHSESEVKEHTFISQTKSSYKKLTSKNKSNTIFSDSYIKHSPNIVQDLNKSREHTSSFNCNFQNGNQEHQTCSIFRARKRASLDINKEKPGASQNEGKTSDKKVVQTFSQKTKKAPLNFNSEKMSRSFRSWKRRKHLKRSRDSSPLKDSGACRIHLQGQTLSNPSLCEDPFTLDEKKTEFRNSGPFAKNVSLSGKEKDNKTSFPLQIKQNCSWNITLTNDNFVEHIVTGSQSKNVTCQATSVVSEKGRGVAVEAEKINEVLIQNGSKNQNVYMKHHDIHPINQYLRKQSHEQTSTITKQKNIIERQMPCEAVSSYINRDSNVTINCERIKLNTEENKPSHFQALGDDISRTVIPSEVLPSAGAFSKSEGQHENFLNISRLQEKTGTYTTNKTKNNHVSDLGLVLCDFEDSFYLDTQSEKIIQQMATENAKLGAKDTNLAAGIMQKSLVQQNSMNSFQKECHIPFPAEQHPLGATKIDHLDLKTVGTMKQSSDSHGVDILTPESPIFHSPILLEENGLFLKKNEVSVTDSQLNSFLQGYQTQETVKPVILLIPQKRTPTGVEGECLPVPETSLNMSDSLLFDSFSDDYLVKEQLPDMQMKEPLPSEVTSNHFSDSLCLQEDLIKKSNVNENQDTHQQLTCSNDESIIFSEMDSVQMVEALDNVDIFPVQEKNHTVVSPRALELSDPVLDEHHQGDQDGGDQDERAEKSKLTGTRQNHSFIWSGASFDLSPGLQRILDKVSSPLENEKLKSMTINFSSLNRKNTELNEEQEVISNLETKQVQGISFSSNNEVKSKIEMLENNANHDETSSLLPRKESNIVDDNGLIPPTPIPTSASKLTFPGILETPVNPWKTNNVLQPGESYLFGSPSDIKNHDLSPGSRNGFKDNSPISDTSFSLQLSQDGLQLTPASSSSESLSIIDVASDQNLFQTFIKEWRCKKRFSISLACEKIRSLTSSKTATIGSRFKQASSPQEIPIRDDGFPIKGCDDTLVVGLAVCWGGRDAYYFSLQKEQKHSEISASLVPPSLDPSLTLKDRMWYLQSCLRKESDKECSVVIYDFIQSYKILLLSCGISLEQSYEDPKVACWLLDPDSQEPTLHSIVTSFLPHELPLLEGMETSQGIQSLGLNAGSEHSGRYRASVESILIFNSMNQLNSLLQKENLQDVFRKVEMPSQYCLALLELNGIGFSTAECESQKHIMQAKLDAIETQAYQLAGHSFSFTSSDDIAEVLFLELKLPPNREMKNQGSKKTLGSTRRGIDNGRKLRLGRQFSTSKDVLNKLKALHPLPGLILEWRRITNAITKVVFPLQREKCLNPFLGMERIYPVSQSHTATGRITFTEPNIQNVPRDFEIKMPTLVGESPPSQAVGKGLLPMGRGKYKKGFSVNPRCQAQMEERAADRGMPFSISMRHAFVPFPGGSILAADYSQLELRILAHLSHDRRLIQVLNTGADVFRSIAAEWKMIEPESVGDDLRQQAKQICYGIIYGMGAKSLGEQMGIKENDAACYIDSFKSRYTGINQFMTETVKNCKRDGFVQTILGRRRYLPGIKDNNPYRKAHAERQAINTIVQGSAADIVKIATVNIQKQLETFHSTFKSHGHREGMLQSDQTGLSRKRKLQGMFCPIRGGFFILQLHDELLYEVAEE

SEQ ID NO: 25 - protein, 772 aa, Homo sapiens
SSSSESLSIIDVASDQNLFQTFIKEWRCKKRFSISLACEKIRSLTSSKTATIGSRFKQASSPQEIPIRDDGFPIKGCDDTLVVGLAVCWGGRDAYYFSLQKEQKHSEISASLVPPSLDPSLTLKDRMWYLQSCLRKESDKECSVVIYDFIQSYKILLLSCGISLEQSYEDPKVACWLLDPDSQEPTLHSIVTSFLPHELPLLEGMETSQGIQSLGLNAGSEHSGRYRASVESILIFNSMNQLNSLLQKENLQDVFRKVEMPSQYCLALLELNGIGFSTAECESQKHIMQAKLDAIETQAYQLAGHSFSFTSSDDIAEVLFLELKLPPNREMKNQGSKKTLGSTRRGIDNGRKLRLGRQFSTSKDVLNKLKALHPLPGLILEWRRITNAITKVVFPLQREKCLNPFLGMERIYPVSQSHTATGRITFTEPNIQNVPRDFEIKMPTLVGESPPSQAVGKGLLPMGRGKYKKGFSVNPRCQAQMEERAADRGMPFSISMRHAFVPFPGGSILAADYSQLELRILAHLSHDRRLIQVLNTGADVFRSIAAEWKMIEPESVGDDLRQQAKQICYGIIYGMGAKSLGEQMGIKENDAACYIDSFKSRYTGINQFMTETVKNCKRDGFVQTILGRRRYLPGIKDNNPYRKAHAERQAINTIVQGSAADIVKIATVNIQKQLETFHSTFKSHGHREGMLQSDQTGLSRKRKLQGMFCPIRGGFFILQLHDELLYEVAEEDVVQVAQIVKNEMESAVKLSVKLKVKVKIGASWGELKDFDV

SEQ ID NO: 26 - protein, 828 aa, Homo sapiens
DKLLLANWGLPKAVLEKYHSFGVKKMFEWQAECLLLGQVLEGKNLVYSAPTSAGKTLVAELLILKRVLEMRKKALFILPFVSVAKEKKYYLQSLFQEVGIKVDGYMGSTSPSRHFSSLDIAVCTIERANGLINRLIEENKMDLLGMVVVDELHMLGDSHRGYLLELLLTKICYITRKSASCQADLASSLSNAVQIVGMSATLPNLELVASWLNAELYHTDFRPVPLLESVKVGNSIYDSSMKLVREFEPMLQVKGDEDHVVSLCYETICDNHSVLLFCPSKKWCEKLADIIAREFYNLHHQAEGLVKPSECPPVILEQKELLEVMDQLRRLPSGLDSVLQKTVPWGVAFHHAGLTFEERDIIEGAFRQGLIRVLAATSTLSSGVNLPARRVIIRTPIFGGRPLDILTYKQMVGRAGRKGVDTVGESILICKNSEKSKGIALLQGSLKPVRSCLQRREGEEVTGSMIRAILEIIVGGVASTSQDMHTYAACTFLAASMKEGKQGIQRNQESVQLGAIEACVMWLLENEFIQSTEASDGTEGKVYHPTHLGSATLSSSLSPADTLDIFADLQRAMKGFVLENDLHILYLVTPMFEDWTTIDWYRFFCLWEKLPTSMKRVAELVGVEEGFLARCVKGKVVARTERQHRQMAIHKRFFTSLVLLDLISEVPLREINQKYGCNRGQIQSLQQSAAVYAGMITVFSNRLGWHNMELLLSQFQKRLTFGIQRELCDLVRVSLLNAQRARVLYASGFHTVADLARANIVEVEVILKNAVPFKSARKAVDEEEEAVEERRNMRTIWVTGRKGLTEREAAALIVEEARMILQQDLVEM

SEQ ID NO: 27 - protein, 388 aa, Artificial Sequence
  SYNTHESIZED
MLGAGQPVPVECRHRLEVAGPRKGPLSPAWMPAYACQRPTPLTHHNTGLSEALEILAEAAGFEGSEGRLLTFCRAASVLKALPSPVTTLSQLQGLPHFGEHSSRVVQELLEHGVCEEVERVRRSERYQTMKLFTQIFGVGVKTADRWYREGLRTLDDLREQPQKLTQQQKAGLQHHQDLSTPVLRSDVDALQQVVEEAVGQALPGATVTLTGGFRRGKLQGHDVDFLITHPKEGQEAGLLPRVMCRLQDQGLILYHQHQESTFEKLRLPSRKVDALDHFERSFLIFRLPQPPGAAVGGSTRPCPSWKAVRVDLVVAPVSQFPFALLGWTGSKLFQRELRRFSRKEKGLWLNSHGLFDPEQKTFFQAASEEDIFRHLGLEYLPPEQRNA

SEQ ID NO: 28 - protein, 388 aa, Artificial Sequence
  SYNTHESIZED
MLGAGQPVPVECRHRLEVAGPRKGPLSPAWMPAYACQRPTPLTHHNTGLSEALEILAEAAGFEGSEGRLLTFCRAASVLKALPSPVTTLSQLQGLPHFGEHSSRVVQELLEHGVCEEVERVRRSERYQTMKLFTQIFGVGVKTADRWYREGLRTLDDLREQPQKLTQQQKAGLQHHQDLSTPVLRSDVDALQQVVEEAVGQALPGATVTLTGGFRRGKLQGHDVDFLITHPKEGQEAGLLPRVMCRLQDQGLILYHQHQESTFEKLRLPSRKVDALDHFERSFCIFRLPQPPGAAVGGSTRPCPSWKAVRVDLVVAPVSQFPFALLGWTGSKLFQRELRRFSRHEKGLWLNSHGLFDPEQKTFFQAASEEDIFRHLGLEYLPPEQRNA

SEQ ID NO: 29 - protein, 388 aa, Artificial Sequence
  SYNTHESIZED
MLGAGQPVPVECRHRLEVAGPRKGPLSPAWMPAYACQRPTPLTHHNTGLSEALEILAEAAGFEGSEGRLLTFCRAASVLKALPSPVTTLSQLQGLPHFGEHSSRVVQELLEHGVCEEVERVRRSERYQTMKLFTQIFGVGVKTADRWYREGLRTLDDLREQPQKLTQQQKAGLQHHQDLSTPVSRSDVDALQQVVEEAVGQALPGATVTLTGGFRRGKLQGHDVDFLITHPKEGQEAGLLPRVMCRLQDQGLILYHQHQESTFEKLRLPSRKVDALDHFERSFCIFRLPQPPGAAVGGSTRPCPSWKAVRVDLVVAPVSQFPFALLGWTGSKLFQRELRRFSRKEKGLWLNSHGLFDPEQKTFFQAASEEDIFRHLGLEYLPPEQRNA

SEQ ID NO: 30 - protein, 388 aa, Artificial Sequence
  SYNTHESIZED
MLGAGQPVPVECRHRLEVAGPRKGPLSPAWMPAYACQRPTPLTHHNTGLSEALEILAEAAGFEGSEGRLLTFCRAASVLKALPSPVTTLSQLQGLPHFGEHSSRVVQELLEHGVCEEVERVRRSERYQTMKLFTQIFGVGVKTADRWYREGLRTLDDLREQPQKLTQQQKAGLQHHQDLSTPVLRSDVDALQQVVEEAVGQALPGATVTLTGGFRRGKEFGHDVDFLITHPKEGQEAGLLPRVMCRLQDQGLILYHQHQESTFEKLRLPSRKVDALDHFERSFCIFRLPQPPGAAVGGSTRPCPSWKAVRVDLVVAPVSQFPFALLGWTGSKLFQRELRRFSRKEKGLWLNSHGLFDPEQKTFFQAASEEDIFRHLGLEYLPPEQRNA

SEQ ID NO: 31 - protein, 388 aa, Artificial Sequence
  SYNTHESIZED
MLGAGQPVPVECRHRLEVAGPRKGPLSPAWMPAYACQRPTPLTHHNTGLSEALEILAEAAGFEGSEGRLLTFCRAASVLKALPSPVTTLSQLQGLPHFGEHSSRVVQELLEHGVCEEVERVRRSERYQTMKLFTQIFGVGVKTADRWYREGLRTLDDLREQPQKLTQQQKAGLQHHQDLSTPVLRSDVDALQQVVEEAVGQALPGATVTLTGGFRRGKEQGHDVDFLITHPKEGQEAGLLPRVMCRLQDQGLILYHQHQESTFEKLRLPSRKVDALDHFERSFCIFRLPQPPGAAVGGSTRPCPSWKAVRVDLVVAPVSQFPFALLGWTGSKLFQRELRRFSRKEKGLWLNSHGLFDPEQKTFFQAASEEDIFRHLGLEYLPPEQRNA

SEQ ID NO: 32 - protein, 388 aa, Artificial Sequence
  SYNTHESIZED
MLGAGQPVPVECRHRLEVAGPRKGPLSPAWMPAYACQRPTPLTHHNTGLSEALEILAEAAGFEGSEGRLLTFCRAASVLKALPSPVTTLSQLQGLPHFGEHSSRVVQELLEHGVCEEVERVRRSERYQTMKLFTQIFGVGVKTADRWYREGLRTLDDLREQPQKLTQQQKAGLQHHQDLSTPVLRSDVDALQQVVEEAVGQALPGATVTLTGGFRRGKLQGHDVDFLITHPKEGQEAGLLPRVMCRLQDQGLILYHQHQESTFEKLRLPSRKVDALDHFERSFCIFRLPQPPGAAVGGSTRPCPSWKAVRVDLVVAPVSQFPFALLGWTGSKQFQRELRRFSRKEKGLWLNSHGLFDPEQKTFFQAASEEDIFRHLGLEYLPPEQRNA

SEQ ID NO: 33 - protein, 388 aa, Artificial Sequence
  SYNTHESIZED
MLGAGQPVPVECRHRLEVAGPRKGPLSPAWMPAYACQRPTPLTHHNTGLSEALEILAEAAGFEGSEGRLLTFCRAASVLKALPSPVTTLSQLQGLPHFGEHSSRVVQELLEHGVCEEVERVRRSERYQTMKLFTQIFGVGVKTADRWYREGLRTLDDLREQPQKLTQQQKAGLQHHQDLSTCVLRSDVDALQQVVEEAVGQALPGATVTLTGGFRRGKLQGHDVDFLITHPKEGQEAGLLPRVMCRLQDQGLILYHQHQESTFEKLRLPSRKVDALDHFERSFCIFRLPQPPGAAVGGSTRPCPSWKAVRVDLVVAPVSQFPFALLGWTGSKLFQRELRRFSRKEKGLWLNSHGLFDPEQKTFFQAASEEDIFRHLGLEYLPPEQRNA

SEQ ID NO: 34 - protein, 388 aa, Artificial Sequence
  SYNTHESIZED
MLGAGQPVPVECRHRLEVAGPRKGPLSPAWMPAYACQRPTPLTHHNTGLSEALEILAEAAGFEGSEGRLLTFCRAASVLKALPSPVTTLSQLQGLPHFGEHSSRVVQELLEHGVCEEVERVRRSERYQTMKLFTQIFGVGVKTADRWYREGLRTLDDLREQPQKLTQQQKAGLQHHQDLSTPVLRSDVDALQQVVEEAVGQALPGATVTLTGGFRRGKLQGHDVDFLITHPKEGQEAGLLPRVMCRLQDQGLILYHQHQESTFEKLRLPSRKVDALDHFERSFCIFRLPQPPGAAVGGSTRPCPSWKAVRVDLVVAPVSQFAFALLGWTGSKLFQRELRRFSRKEKGLWLNSHGLFDPEQKTFFQAASEEDIFRHLGLEYLPPEQRNA

SEQ ID NO: 35 - protein, 388 aa, Artificial Sequence
  SYNTHESIZED
MLGAGQPVPVECRHRLEVAGPRKGPLSPAWMPAYACQRPTPLTHHNTGLSEALEILAEAAGFEGSEGRLLTFCRAASVLKALPSPVTTLSQLQGLPHFGEHSSRVVQELLEHGVCEEVERVRRSERYQTMKLFTQIFGVGVKTADRWYREGLRTLDDLREQPQKLTQQQKAGLQHHQDLSTPVLRSDVDALQQVVEEAVGQALPGATVTLTGGFRRGKLFGHDVDFLITHPKEGQEAGLLPRVMCRLQDQGLILYHQHQESTFEKLRLPSRKVDALDHFERSFCIFRLPQPPGAAVGGSTRPCPSWKAVRVDLVVAPVSQFPFALLGWTGSKLFERELRRFSRKEKGLWLNSHGLFDPEQKTFFQAASEEDIFRHLGLEYLPPEQRNA

SEQ ID NO: 36 - protein, 388 aa, Artificial Sequence
  SYNTHESIZED
MLGAGQPVPVECRHRLEVAGPRKGPLSPAWMPAYACQRPTPLTHHNTGLSEALEILAEAAGFEGSEGRLLTFCRAASVLKALPSPVTTLSQLQGLPHFGEHSSRVVQELLEHGVCEEVERVRRSERYQTMKLFTQIFGVGVKTADRWYREGLRTLDDLREQPQKLTQQQKAGLQHHQDLSTPVLRSDVDALQQVVEEAVGQALPGATVTLTGGFRRGKLFGHDVDFLITHPKEGQEAGLLPRVMCRLQDQGLILYHQHQESTFEKLRLPSRKVDALDHFERSFCIFRLPQPPGAAVGGSTRPCPSWKAVRVDLVVAPVSQFPFALLGWTGSKLFQRELRRFSRKEKGLWLNSHGLFDPEQKTFFQAASEEDIFRHLGLEYLPPEQRNA

SEQ ID NO: 37 - protein, 388 aa, Artificial Sequence
  SYNTHESIZED
MLGAGQPVPVECRHRLEVAGPRKGPLSPAWMPAYACQRPTPLTHHNTGLSEALEILAEAAGFEGSEGRLLTFCRAASVLKALPSPVTTLSQLQGLPHFGEHSSRVVQELLEHGVCEEVERVRRSERYQTMKLFTQIFGVGVKTADRWYREGLRTLDDLREQPQKLTQQQKAGLQHHQDLSTPVLRSDVDALQQVVEEAVGQALPGATVTLTGGFRRGKLQGHDVDFLITHPKEGQEAGLLPRVMCRLQDQGLILYHQHQESTFEKLRLPSRKVDALDHFERSFCIFRLPQPPGAAVGGSTRPCPSWKAVRVDLVVAPVSQFPFALLGWTGSKLFERELRRFSRKEKGLWLNSHGLFDPEQKTFFQAASEEDIFRHLGLEYLPPEQRNA

SEQ ID NO: 38 - protein, 388 aa, Artificial Sequence
  SYNTHESIZED
MLGAGQPVPVECRHRLEVAGPRKGPLSPAWMPAYACQRPTPLTHHNTGLSEALEILAEAAGFEGSEGRLLTFCRAASVLKALPSPVTTLSQLQGLPHFGEHSSRVVQELLEHGVCEEVERVRRSERYQTMKLFTQIFGVGVKTADRWYREGLRTLDDLREQPQKLTQQQKAGLQHHQDLSTPVLRSDVDALQQVVEEAVGQALPGATVTLTGGFRRGKLQGHDVDFLITHPKEGQEAGLLPRVMCRLQDQGLILYHQHQESTFEKLRLPSRKVDALDHFERSFCIFRLPQPPGAAVGGSTRPCPSWKAVRVDLVVAPVSQFPFALLGWTGSKLFQRELRRFSTHEKGLWLNSHGLFDPEQKTFFQAASEEDIFRHLGLEYLPPEQRNA

SEQ ID NO: 39 - protein, 388 aa, Artificial Sequence
  SYNTHESIZED
MLGAGQPVPVECRHRLEVAGPRKGPLSPAWMPAYACQRPTPLTHHNTGLSEALEILAEAAGFEGSEGRLLTFCRAASVLKALPSPVTTLSQLQGLPHFGEHSSRVVQELLEHGVCEEVERVRRSERYQTMKLFTQIFGVGVKTADRWYREGLRTLDDLREQPQKLTQQQKAGLQHHQDLSTPVLRSDVDALQQVVEEAVGQALPGATVTLTGGFRRGKLQGHDVDFLITHPKEGQEAGLLPRVMCRLQDQGLILYHQHQESTFEKLRLPSRKVDALDHFERSFCIFRLPQPPGAAVGGSTRPCPSWKAVRVDLVVAPVSQFPFALLGWTGSKLFQRELRRFSTKEKGLWLNSHGLFDPEQKTFFQAASEEDIFRHLGLEYLPPEQRNA

SEQ ID NO: 40 - protein, 252 aa, Sarciphilus harrisii
KWYRMGFRTLSKIQSDKSLKFTKMQKAGFLYYEDLISCVSKAEADAVSLIVKEAVWTFLPDALITITGGFRRGKEFGHDVDFLITSPGGEKEQVDQLLQKVTNLWEKQGLLLYYDLMESTFEDLKLPSRKVDALDHFQKCFLILKLYCQRGDRSKWEGPEGSNGLQTKNWKAIRVDLVVCPYDRYAYALLGWSGSRQFERDLRRYATHEKKMMLDNHALYDKTKRTFLKAESEEEIFSHLGLEYIEPWERNA

SEQ ID NO: 41 - protein, 245 aa, Homo sapiens
KWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 42 - protein, 242 aa, Homo sapiens
RWYREGLRTLDDLREQPQKLTQQQKAGLQHHQDLSTPVLRSDVDALQQVVEEAVGQALPGATVTLTGGFRRGKLQGHDVDFLITHPKEGQEAGLLPRVMCRLQDQGLILYHQHQHSCCESPTRLAQQSHMDAFERSFCIFRLPQPPGAAVGGSTRPCPSWKAVRVDLVVAPVSQFPFALLGWTGSKLFQRELRRFSRKEKGLWLNSHGLFDPEQKTFFQAASEEDIFRHLGLEYLPPEQRNA

SEQ ID NO: 43 - protein, 225 aa, Homo sapiens [sequence omitted]

SEQ ID NO: 44 - protein, 220 aa, Homo sapiens
KFVDEGIKTLEDLRKNDKLNHHQRIGLKYFGDFEKRIPREEMLQMQDIVLNEVKKVDSEYIATVCGSFRRGAESSGDMDVLLTHPSFTSESTKQPKLLHQVVEQLQKVHFITDTKGETKFMGVCQLPSKNDEKEYPHRRIDIRLIPKDQYYCGVLYFTGSDIFNKNMRAHALEKGFTINEYTIRPLGVTGVAGEPLPVDSEKDIFDYIQWKYREPKDRSE

SEQ ID NO: 45 - protein, 172 aa, African swine fever virus
SKNIVAVGSLRREEKMLNDVDLLIIVPEKKLLKHVLPNIRIKGLSFSVKTLIQGKKIVNHLRSRLAFEYNGQLIKILVCGERKCVLFIEWEKKTYQLDLFTALAEEKPYAIFHFTGPVSYLIRIRAALKKKNYKLNQYGLFKNQTLVPLKITTEKELIKELGFTYRIPKKRL

SEQ ID NO: 46 - protein, 380 aa, Homo sapiens
IGSRFKQASSPQEIPIRDDGFPIKGCDDTLVVGLAVCWGGRDAYYFSLQKEQKHSEISASLVPPSLDPSLTLKDRMWYLQSCLRKESDKECSVVIYDFIQSYKILLLSCGISLEQSYEDPKVACWLLDPDSQEPTLHSIVTSFLPHELPLLEGMETSQGIQSLGLNAGSEHSGRYRASVESILIFNSMNQLNSLLQKENLQDVFRKVEMPSQYCLALLELNGIGFSTAECESQKHIMQAKLDAIETQAYQLAGHSFSFTSSDDIAEVLFLELKLPPNREMKNQGSKKTLGSTRRGIDNGRKLRLGRQFSTSKDVLNKLKALHPLPGLILEWRRITNAITKVVFPLQREKCLNPFLGMERIYPVSQSHTATGRITFTEPNI

SEQ ID NO: 47 - protein, 310 aa, Homo sapiens
VLKALPSPVTTLSQLQGLPHFGEHSSRVVQELLEHGVCEEVERVRRSERYQTMKLFTQIFGVGVKTADRWYREGLRTLDDLREQPQKLTQQQKAGLQHHQDLSTPVLRSDVDALQQVVEEAVGQALPGATVTLTGGFRRGKLQGHDVDFLITHPKEGQEAGLLPRVMCRLQDQGLILYHQHQHSCCESPTRLAQQSHMDAFERSFCIFRLPQPPGAAVGGSTRPCPSWKAVRVDLVVAPVSQFPFALLGWTGSKLFQRELRRFSRKEKGLWLNSHGLFDPEQKTFFQAASEEDIFRHLGLEYLPPEQRNA

SEQ ID NO: 48 - protein, 313 aa, Homo sapiens
VLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 49 - protein, 320 aa, Sarciphilus harrisii
VLKCLPFAIVSMKDAEGLPWIGDEVKGIMEEIIEDGQSLEVQAVLNDERYQAFKLFTSVFGVGLKTAEKWYRMGFRTLSKIQSDKSLKFTKMQKAGFLYYEDLISCVSKAEADAVSLIVKEAVWTFLPDALITITGGFRRGKEFGHDVDFLITSPGGEKEQVDQLLQKVTNLWEKQGLLLYYDLMESTFEDLKLPSRKVDALDHFQKCFLILKLYCQRGDRSKWEGPEGSNGLQTKNWKAIRVDLVVCPYDRYAYALLGWSGSRQFERDLRRYATHEKKMMLDNHALYDKTKRTFLKAESEEEIFSHLGLEYIEPWERNA

SEQ ID NO: 50 - protein, 290 aa, Homo sapiens
ALKSFHKPVTSYQEACSIPGIGKRMAEKIIEILESGHLRKLDHISESVPVLELFSNIWGAGTKTAQMWYQQGFRSLEDIRSQASLTTQQAIGLKHYSDFLERMPREEATEIEQTVQKAAQAFNSGLLCVACGSYRRGKATCGDVDVLITHPDGRSHRGFSRLLDSLRQEGFLTDDLVSQEENGQQQKYLGVCRLPGPGRRHRRLDIIVVPYSEFACALLYFTGSAHFNRSMRALAKTKGMSLSEHALSTAVVRNTHGCKVGPGRVLPTPTEKDVFRLLGLPYREPAERDW

SEQ ID NO: 51 - protein, 291 aa, Homo sapiens
VIAKYPHKIKSGAEAKKLPGVGTKIAEKIDEFLATGKLRKLEKIRQDDTSSSINFLTRVSGIGPSAARKFVDEGIKTLEDLRKNEDKLNHHQRIGLKYFGDFEKRIPREEMLQMQDIVLNEVKKVDSEYIATVCGSFRRGAESSGDMDVLLTHPSFTSESTKQPKLLHQVVEQLQKVHFITDTLSKGETKFMGVCQLPSKNDEKEYPHRRIDIRLIPKDQYYCGVLYFTGSDIFNKNMRAHALEKGFTINEYTIRPLGVTGVAGEPLPVDSEKDIFDYIQWKYREPKDRSE

(19) United States
(12) Patent Application Publication
(10) Pub. No.: US 2019/0078065 A1
(43) Pub. Date: Mar. 14, 2019

(54) MODIFIED DNA POLYMERASES

(71) Applicants: SIGMA-ALDRICH CO. LLC, St. Louis, MO (US); Merck Patent GmbH, Darmstadt (DE)

(72) Inventors: Thomas Baiga, Darmstadt (DE); Michael Anderson Burley, Darmstadt (DE); Alexander Smith, St. Louis, MO (US)

(21) Appl. No.: 16/125,460

(22) Filed: Sep. 7, 2018

Related U.S. Application Data
(60) Provisional application No. 62/556,083, filed on Sep. 8, 2017.
(60) Provisional application No. 62/556,090, filed on Sep. 8, 2017.

(51) Int. Cl. C12N 9/12; C12N 15/10; C12N 15/52

(52) U.S. Cl. CPC C12N 9/1252; C12N 15/1068; C12Y 207/07001; C12N 15/52; C12N 15/1096

(57) ABSTRACT

Modified X family DNA polymerases engineered to be capable of incorporating 3′-O-blocked nucleotide 5′-triphosphates during template-independent polynucleotide synthesis, and methods for synthesizing polynucleotides using said modified X family DNA polymerases.

### CROSS REFERENCE TO RELATED APPLICATIONS

This application claims priority to U.S. Provisional Application Ser. No. 62/556,083, filed Sep. 8, 2017, and U.S. Provisional Application Ser. No. 62/556,090, filed Sep. 8, 2017, and the disclosure of each is hereby incorporated by reference in its entirety.

### SEQUENCE LISTING

This application contains a Sequence Listing that has been submitted in ASCII format via EFS-Web and is hereby incorporated by reference in its entirety. The ASCII copy, created on Sep. 6, 2018, is named 604654_SequenceListing_ST25.txt, and is 168 kilobytes in size.

### FIELD

The present disclosure generally relates to engineered DNA X family DNA polymerases that are capable of incorporating 3′-O-blocked nucleotides during template-independent polynucleotide synthesis.

### BACKGROUND

The synthesis and assembly of gene length DNA represents a significant bottleneck in modern biology. Oligonucleotide synthesis technologies are still based on chemistries developed in the 1970s and 1980s. In contrast, new and better DNA sequencing technologies have dramatically decreased the cost and increased the speed of sequencing. Thus, there is a need for new and improved polynucleotide synthesis methods that can quickly generate oligonucleotides or polynucleotides without the use of harsh chemical solvents. To accomplish this, there is a need for engineered DNA polymerases that can accommodate nucleotides comprising blocking groups and catalyze template-independent polynucleotide synthesis.

### SUMMARY

Among the various aspects of the present disclosure are modified X family DNA polymerases, which are engineered to comprise one or more mutations. In particular, the modified X family DNA polymerase comprises SEQ ID NO:1 inserted into a loop 1 region.

Another aspect of the present disclosure encompasses methods for synthesizing a polynucleotide. The methods comprise (a) providing an entity comprising a free hydroxyl group; (b) contacting the free hydroxyl group with a nucleotide 5′-triphosphate comprising a removable 3′-O-blocking group in the presence of a modified X family DNA, as disclosed herein, and in the absence of a nucleic acid template to form a linked nucleotide comprising a removable 3′-O-blocking group; (c) contacting the linked nucleotide comprising the removable 3′-O-blocking group with a deblocking agent to remove the removable 3′-O-blocking group; and (d) repeating steps (b) and (c) to yield the polynucleotide.

Other aspects and iterations of the disclosure are detailed below.

### BRIEF DESCRIPTION OF THE DRAWINGS

FIG. 1 presents a multiple sequence alignment generated with CLUSTAL Omega (1.2.4). Shown are the amino acid sequences of relevant portions of Sarciphilus harrisii terminal deoxynucleotidyl transferase (TdT) (G3VQ54; SEQ ID NO:40), human TdT (P04053; SEQ ID NO:41), human DNA polM (Q9NP87; SEQ ID NO:42), human DNA polL (Q9UGP5; SEQ ID NO:43), human DNA polB (P06746; SEQ ID NO:404, and African swine fever virus (ASFV) DNA pol X (P42494; SEQ ID NO:45). Functional motifs are boxed and identified at the right.

FIG. 2 shows a multiple sequence alignment generated with CLUSTAL Omega (1.2.4). Shown are the amino acid sequences of relevant portions of human DNA polQ (O75417; SEQ ID NO:46), ASFV DNA polX (P42494; SEQ ID NO:22), human DNA polM (Q9NP87; SEQ ID NO:47), human TdT (Hs Dntt; P04053; SEQ ID NO:48), S. harrisii TdT (G3VQ54; SEQ ID NO:49), human DNA polL (Q9UGP5; SEQ ID NO:50), and human DNA polB (P06746; SEQ ID NO:51).

FIG. 3 presents a schematic diagram of a polymerase-mediated, template-independent polynucleotide synthesis method.

FIG. 4 shows a schematic diagram of a polymerase-mediated, template-independent, initiator sequence-independent polynucleotide synthesis method. As detailed below, L is a linker, PC is a cleavable group, W is blocking group, and B is a base or analog thereof.

FIG. 5 illustrates template-independent incorporation of 3′-O-carbamate or ester blocked nucleotides by the modified X family DNA polymerase, Hs PolM-Lp1.

FIG. 6 shows multiple cycles of incorporation (and deblocking) by Hs PolM-Lp1.

### DETAILED DESCRIPTION

The present disclosure provides modified X family DNA polymerases that are engineered to accommodate 3′-O-blocked nucleotide 5′-triphosphates and incorporate 3′-O-blocked nucleotides during template-independent polynucleotide synthesis. The modified X family DNA polymerases are engineered to comprise one or more mutations in regions of the protein identified by sequence alignments and computer modeling technology. Also provided herein are methods for modifying the DNA polymerases and methods for synthesizing polynucleotides using the modified X family DNA polymerases and 3′-O-blocked nucleotide 5′-triphosphates.

### (I) Modified X Family DNA Polymerases

Provided herein are modified X family DNA polymerases that have been engineered to contain one or more mutations. The one or more mutations can be insertions of one or more amino acids, deletions of one or more amino acids, and/or substitutions of one or more amino acids. As such, the modified X family DNA polymerases are capable of accommodating 3′-O-reversibly blocked nucleotide 5′-triphosphates, have increased activity in the presence of 3′-O-reversibly blocked nucleotide 5′-triphosphates, and/or are capable of synthesizing polynucleotides in the absence of a nucleic acid template. In general, the modified X family DNA polymerase is other than a terminal deoxynucleotidyl transferase (TdT).

The modified X family DNA polymerase can be derived from an X family DNA polymerase of eukaryotic, viral, archaeal, or bacterial origin. For example, the modified X family DNA polymerase can be derived from DNA polymerase beta (DNA pol β), DNA polymerase lambda (DNA pol X), DNA polymerase mu (DNA pol μ), DNA polymerase theta (DNA pol θ), DNA polymerase X, homologs, orthologs, or paralogs thereof. In particular embodiments, the modified X family DNA polymerase can be derived from a mammalian X family DNA polymerase (e.g., human, primate, mouse, rat, bovine, and the like) or a vertebrate X family DNA polymerase (e.g., frog, fish, birds, etc.).

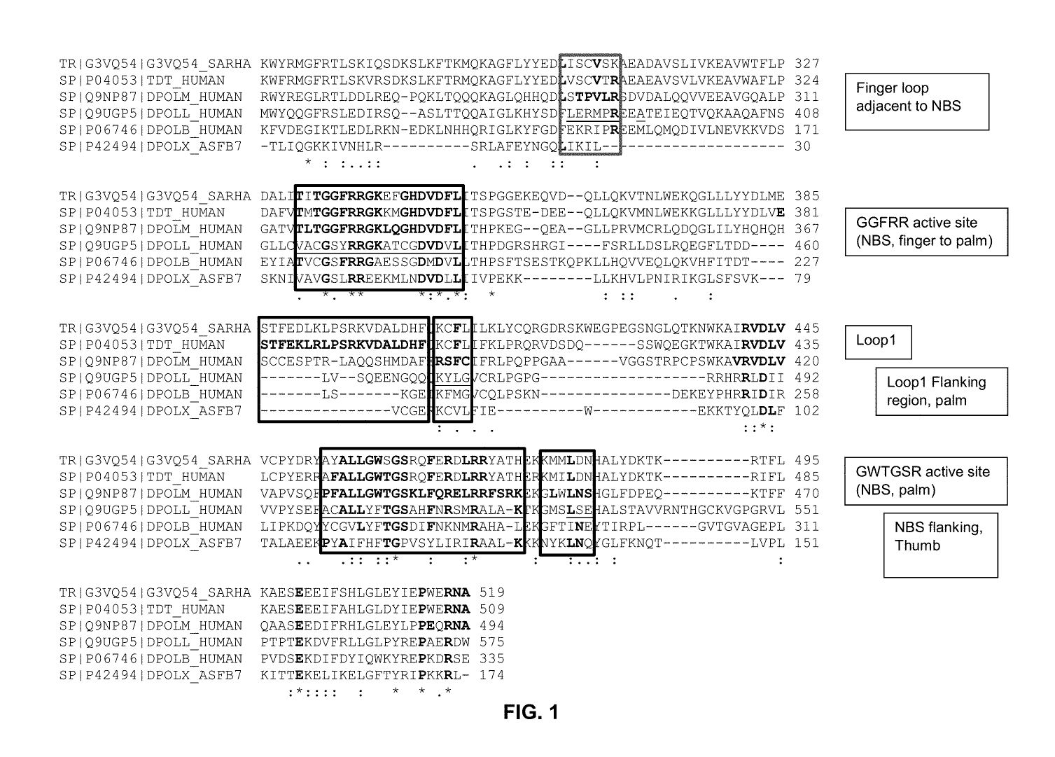

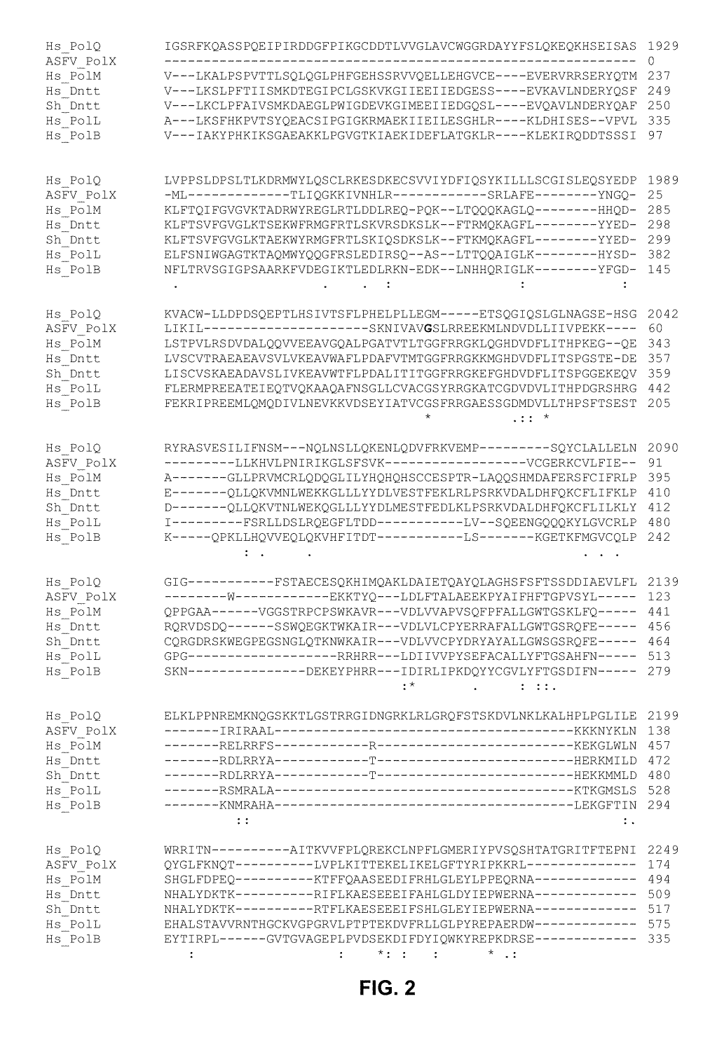

In some embodiments, the X family DNA polymerase can be derived from human DNA polymerase beta (UniprotKB No. P06746, DPOLB_Human) or an ortholog thereof. In other embodiments, the X family DNA polymerase can be derived from human DNA polymerase lambda (UniprotKB No. Q9UGP5, DPOLL_Human) or an ortholog thereof. In still other embodiments, the X family DNA polymerase can be derived from human DNA polymerase mu (UniprotKB No. Q9NP87, DPOLM_Human) or an ortholog thereof. In other embodiments, the X family DNA polymerase can be derived from human DNA polymerase theta (UniprotKB No. O75417, DPOLQ_Human) or an ortholog thereof. In yet other embodiments, the X family DNA polymerase can be derived from DNA polymerase X (UniprotKB No. P42494, DPOLX_ASFB7) or an ortholog thereof. The locations of conserved functional motifs within these polymerases are indicated with boxes in the sequence alignment presented in FIG. 1.

In some embodiments, the one or more mutations in the modified X family DNA polymerase can be an insertion of a sequence comprising ESTFEKLRLPSRKVDALDHF (SEQ ID NO:1) into a loop 1 region of the X family DNA polymerase. For example, SEQ ID NO:1 can be inserted into or substituted with amino acids at positions 231-233 of human DNA polymerase beta, positions 462-470 of human DNA polymerase lambda, positions 367-385 of human DNA polymerase mu, positions 2071-2080 of human DNA polymerase theta, positions 82-84 of ASFV DNA polymerase X, ortholog thereof, or paralog thereof.

In other embodiments, the one or more mutations in the modified X family DNA polymerase can comprise a truncation at the N-terminal end and/or the C-terminal end. The truncation can encompass a portion or all of the sequence N-terminal to the finger loop adjacent to NBS motif and/or the truncation can encompass a portion or all of the sequence C-terminal to palm NBS flanking region motif. For example, an N-terminal truncation can comprise any number of amino acids up to position 145 of human DNA polymerase beta, up to position 382 of human DNA polymerase lambda, up to position 285 of human DNA polymerase mu, up to position 1989 of human DNA polymerase theta, up to position 25 of ASFV DNA polymerase X, ortholog thereof, or paralog thereof. A C-terminal truncation can comprise any number of amino acids from position 296 of human DNA polymerase beta, from position 530 of human DNA polymerase lambda, from position 459 of human DNA polymerase mu, from position 2201 of human DNA polymerase theta, from position 140 of ASFV DNA polymerase X, ortholog thereof, or paralog thereof.

In still other embodiments, the one or more mutations in the modified X family DNA polymerase can be within a finger loop adjacent to nucleotide binding site (NBS) motif located at positions 146-152 of human DNA polymerase beta, positions 383-389 of human DNA polymerase lambda, positions 286-292 of human DNA polymerase mu, positions 1990-1995 of human DNA polymerase theta, positions 26-30 of ASFV DNA polymerase X, ortholog thereof, or paralog thereof. In some iterations, the finger loop adjacent to NBS motif of the modified X family DNA polymerase can comprise sequence L-X-X₁-X-V-X-X (SEQ ID NO:2), wherein X is any amino acid and X₁ is Ser or Thr. For example, the amino acid at position 1 of SEQ ID NO:2 of the finger loop adjacent to NBS motif of the modified X family DNA polymerase can be or can be changed to Leu, the amino acid at position 3 of the finger loop adjacent to NBS motif of the modified X family DNA polymerase can be or can be changed to Thr or Ser, and/or the amino acid at position 5 of the finger loop adjacent to NBS motif of the modified X family DNA polymerase can be or can be changed to Val.

In other embodiments, the one or more mutations in the modified X family DNA polymerase can be within a finger to palm NBS motif located at positions 176-194 of human DNA polymerase beta, positions 413-431 of human DNA polymerase lambda, positions 316-334 of human DNA polymerase mu, positions 2019-2032 of human DNA polymerase theta, positions 35-53 of ASFV DNA polymerase X, ortholog thereof, or paralog thereof. In some iterations, the finger to palm NBS motif of the modified X family DNA polymerase can comprise sequence X₁-X-X₁-G-G-X₃-X₂-X₂-G-X₁-X-X-G-H-D-V-D-X₃-L (SEQ ID NO:3), wherein X is any amino acid, X₁ is Ser or Thr, X₂ is Arg or Lys, and X₃ is Phe or Tyr. For example, the amino acid at position 1 of SEQ ID NO:3 of the finger to palm NBS motif can be or can be changed to Thr or Set, the amino acid at position 2 of SEQ ID NO:3 of the finger to palm NBS motif can be or can be changed to Thr or Ser, the amino acid at position 4 of SEQ ID NO:3 of the finger to palm NBS motif can be or can be changed to Gly, the amino acid at position 5 of SEQ ID NO:3 of the finger to palm NBS motif can be or can be changed to Gly, the amino acid at position 6 of SEQ ID NO:3 of the finger to palm NBS motif can be or can be changed to Phe or Tyr, the amino acid at position 7 of SEQ ID NO:3 of the finger to palm NBS motif can be or can be changed to Arg or Lys, the amino acid at position 8 of SEQ ID NO:3 of the finger to palm NBS motif can be or can be changed to Arg or Lys, the amino acid at position 9 of SEQ ID NO:3 of the finger to palm NBS motif can be or can be changed to Gly, the amino acid at position 10 of SEQ ID NO:3 of the finger to palm NBS motif can be or can be changed to Lys or Arg, the amino acid at position 10 of SEQ ID NO:3 of the finger to palm NBS motif can be or can be changed to Lys or Arg, the amino acid at position 13 of SEQ ID NO:3 of the finger to palm NBS motif can be or can be changed to Gly, the amino acid at position 14 of SEQ ID NO:3 of the finger to palm NBS motif can be or can be changed to His, the amino acid at position 15 of SEQ ID NO:3 of the finger to palm NBS motif can be or can be changed to Asp, the amino acid at position 16 of SEQ ID NO:3 of the finger to palm NBS motif can be or can be changed to Val, the amino acid at position 17 of SEQ ID NO:3 of the finger to palm NBS motif can be or can be changed to Asp, the amino acid at position 18 of SEQ ID NO:3 of the finger to palm NBS motif can be or can be changed to Phe or Tyr, and/or the amino acid at position 19 of SEQ ID NO:3 of the finger to palm NBS motif can be or can be changed to Leu.

In still other embodiments, the one or more mutations in the modified X family DNA polymerase can be within a Loop1 flanking region motif located at positions 233-237 of human DNA polymerase beta, positions 471-475 of human DNA polymerase lambda, positions 386-390 of human DNA polymerase mu, positions 2081-2085 of human DNA polymerase theta, positions 84-88 of ASFV DNA polymerase X, ortholog thereof, or paralog thereof. The Loop1 flanking region motif of the modified X family DNA polymerase can comprise sequence Q-X-X-X₃-X (SEQ ID NO:4), wherein X is any amino acid and X₃ is Phe or Tyr. For example, the amino acid at position 1 of SEQ ID NO:4 of the Loop1 flanking region motif can be or can be changed to Gin, and/or the amino acid at position 4 of the Loop1 flanking region motif can be or can be changed to Phe or Tyr.

In further embodiments, the one or more mutations in the modified X family DNA polymerase can be within a Loop1 flanking in palm motif located at positions 253-258 of human DNA polymerase beta, positions 487-492 of human DNA polymerase lambda, positions 415-420 of human DNA polymerase mu, positions 2105-2113 of human DNA polymerase theta, positions 97-102 of ASFV DNA polymerase X, ortholog thereof, or paralog thereof. The Loop1 flanking in palm motif in the modified X family DNA polymerase can comprise sequence X-X₂-V-D-L-V (SEQ ID NO:5), wherein X is any amino acid and X₂ is Arg or Lys. For example, the amino acid at position 2 of SEQ ID NO:5 of the Loop1 flanking in palm motif can be or can be changed to Arg or Lys, the amino acid at position 3 of SEQ ID NO:5 of the Loop1 flanking in palm motif can be or can be changed to Val, the amino acid at position 4 of SEQ ID NO:5 of the Loop1 flanking in palm motif can be or can be changed to Asp, the amino acid at position 5 of SEQ ID NO:5 of the Loop1 flanking in palm motif can be or can be changed to Leu, and/or the amino acid at position 6 of SEQ ID NO:5 of the Loop1 flanking in palm motif can be or can be changed to Val.

In yet other embodiments, the one or more mutations in the modified X family DNA polymerase can be within a palm NBS motif located at positions 266-287 of human DNA polymerase beta, positions 500-521 of human DNA polymerase lambda, positions 428-450 of human DNA polymerase mu, positions 2121-2192 of human DNA polymerase theta, positions 110-131 of ASFV DNA polymerase X, ortholog thereof, or paralog thereof. The palm NBS motif of the modified X family DNA polymerase can comprise sequence X-X₃-A-L-L-G-W-X₁-G-X₁-X₂-X-X₃-X-X₂-X-L-X₂-X₂-X₃-X-X-X (SEQ ID NO:6), wherein X is any amino acid, X₁ is Ser or Thr, X₂ is Arg or Lys, and X₃ is Phe or Tyr. For example, the amino acid at position 2 of SEQ ID NO:6 of the palm NBS motif can be or can be changed to Phe or Tyr, the amino acid at position 3 of SEQ ID NO:6 of the palm NBS motif can be or can be changed to Ala, the amino acid at position 4 of SEQ ID NO:6 of the palm NBS motif can be or can be changed to Leu, the amino acid at position 5 of SEQ ID NO:6 of the palm NBS motif can be or can be changed to Leu, the amino acid at position 6 of SEQ ID NO:6 of the palm NBS motif can be or can be changed to Leu, the amino acid at position 7 of SEQ ID NO:6 of the palm NBS motif can be or can be changed to Trp, the amino acid at position 8 of SEQ ID NO:6 of the palm NBS motif can be or can be changed to Thr or Ser, the amino acid at position 9 of SEQ ID NO:6 of the palm NBS motif can be or can be changed to Gly, the amino acid at position 10 of SEQ ID NO:6 of the palm NBS motif can be or can be changed to Thr or Ser, the amino acid at position 11 of SEQ ID NO:6 of the palm NBS motif can be or can be changed to Arg or Lys, the amino acid at position 13 of SEQ ID NO:6 of the palm NBS motif can be or can be changed to Phe or Try, the amino acid at position 15 of SEQ ID NO:6 of the palm NBS motif can be or can be changed to Arg or Lys, the amino acid at position 17 of SEQ ID NO:6 of the palm NBS motif can be or can be changed to Leu, the amino acid at position 18 of SEQ ID NO:6 of the palm NBS motif can be or can be changed to Arg or Lys, the amino acid at position 19 of SEQ ID NO:6 of the palm NBS motif can be or can be changed to Arg or Lys, and/or the amino acid at position 20 of SEQ ID NO:6 of the palm NBS motif can be Phe or Try.

In alternate embodiments, the one or more mutations in the modified X family DNA polymerase can be within a palm NBS flanking region motif located at positions 290-295 of human DNA polymerase beta, positions 524-529 of human DNA polymerase lambda, positions 453-458 of human DNA polymerase mu, positions 2195-2200 of human DNA polymerase theta, positions 134-139 of ASFV DNA polymerase X, ortholog thereof, or paralog thereof. The palm NBS flanking region motif of the modified X family DNA polymerase can comprise sequence X-X-X-L-X-X (SEQ ID NO:7), wherein X is any amino acid. For example, the amino acid at position 4 of SEQ ID NO:7 of the palm NBS flanking region motif can be or can be changed to Leu.

In still other embodiments, the one or more mutations in the modified X family DNA polymerase can comprise point mutations in which a specific amino acid is changed to another amino acid. The amino acid substitutions can be conservative (i.e., substitution with amino acids having similar chemical properties such as polarity, charge, and the like), or the amino acid substitutions can be nonconservative (i.e., substitution with any other amino acid). Examples of conservative substitutions are shown below.

| | |
|---|---|
| Polar, positive | His (H) |
| | Lys (K) |
| | Arg (R) |
| Polar, negative | Asp (D) |
| | Glu (E) |
| Polar, neutral | Ser (S) |
| | Thr (T) |
| | Asn (N) |
| | Gln (Q) |
| Non-polar, aliphatic | Ala (A) |
| | Val (V) |
| | Leu (L) |
| | Ile (I) |
| | Met (M) |
| Non-polar, aromatic | Phe (F) |
| | Tyr (Y) |
| | Trp (W) |

Non-limiting examples of positions that can be substituted with another amino acid include P289, L291, L362, Q327, C390, P428, L439, Q441, R449, and/or K450 of human DNA polymerase mu or an equivalent residue in another X family DNA polymerase, ortholog, or paralog thereof. In specific embodiments, the point mutation can be P289C, L291S, L362E, Q327F, C390L, P428A, L439Q, Q441E, R449T, and/or K450H of human DNA polymerase mu or an equivalent residue in another X family DNA polymerase, ortholog, or paralog thereof.

The number of mutations in the modified X family DNA polymerase can and will vary depending upon the identity or source of the polymerase and/or the desired activity of the modified polymerase. In general, the modified X family DNA polymerase will comprise the smallest number of mutations needed to modify the nucleotide binding site and/or the catalytic active site such that the modified polymerase can synthesize single-stranded polynucleotides with 3′-O-blocked nucleotide 5′-triphosphates in the absence of a nucleic acid template. In some embodiments, the modified X family DNA polymerase can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mutations, wherein the mutation can be an amino acid substitution, deletion, and/or insertion.

In some embodiments, the modified X family DNA polymerase can further comprise at least one marker domain and/or purification tag. Non-limiting examples of marker domains include fluorescent proteins, purification tags, and epitope tags. In some embodiments, the marker domain can be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1,), blue fluorescent proteins (e.g. EBFP, EBFP2, Azurite, mKalamal, GFPuv, Sapphire, T-sapphire,), cyan fluorescent proteins (e.g. ECFP, Cerulean, CyPet, AmCyanl, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. Examples of purification tags include, without limit, poly-His, FLAG, HA, tandem affinity purification (TAP), glutathione-S-transferase (GST), chitin binding protein (CBP), maltose binding protein, thioredoxin (TRX), poly(NANP), myc, AcV5, AU1, AU5, E, ECS, E2, nus, Softag 1, Softag 3, Strep, SBP, Glu-Glu, HSV, KT3, S, S1, T7, V5, VSV-G, biotin carboxyl carrier protein (BCCP), and calmodulin. The marker domain and/or purification can be located at the N-terminal end and/or the C-terminal end of the modified polymerase.

Specific Modified X Family DNA Polymerases

In some embodiments, the modified X family DNA polymerase can comprise an insertion or swap of SEQ ID NO:1 into a Loop 1 motif or corresponding region of the polymerase. For example, the modified X family DNA polymerase can have at least about 80%, 82%, 84%, 86%, 88%, 90%, 91%, 92%, 93%, 94%, 95%, 96%, 97%, 98%, or 99% sequence identity to SEQ ID NO:15, SEQ ID NO:18, SEQ ID NO:21, or SEQ ID NO:23. In certain iterations, the modified X family DNA polymerase can have at least 90% or at least 95% sequence identity to SEQ ID NO:15, SEQ ID NO:18, SEQ ID NO:21, or SEQ ID NO:23. In other iterations, the modified X family DNA polymerase can consist of SEQ ID NO:15, SEQ ID NO:18, SEQ ID NO:21, or SEQ ID NO:23.

In other embodiments, the modified X family DNA polymerase can comprise a N-terminal truncation and an insertion or swap of SEQ ID NO:1 into a Loop 1 motif or corresponding region. For example, the modified X family DNA polymerase can have at least about 80%, 82%, 84%, 86%, 88%, 90%, 91%, 92%, 93%, 94%, 95%, 96%, 97%, 98%, or 99% sequence identity to SEQ ID NO:16 or SEQ ID NO:19. In some aspects, the modified X family DNA polymerase can have at least 90% or at least 95% sequence identity to SEQ ID NO:16 or SEQ ID NO:19. In other embodiments, the modified X family DNA polymerase can have less than 400 amino acids and at least about 90% or at least about 95% sequence identity to SEQ ID NO:16. In certain embodiments, the modified X family DNA polymerase can consist of SEQ ID NO:16 or SEQ ID NO:19.

In still further embodiments, the modified X family DNA polymerase can comprise a N-terminal truncation, an insertion or swap of SEQ ID NO:1 into a Loop 1 motif or corresponding region, and at least one point mutation. For example, the modified X family DNA polymerase can have at least about 80%, 82%, 84%, 86%, 88%, 90%, 91%, 92%, 93%, 94%, 95%, 96%, 97%, 98%, or 99% sequence identity to SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, or SEQ ID NO:39. In certain embodiments, the modified X family DNA polymerase can have at least 90% or at least 95% sequence identity to SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, or SEQ ID NO:39. In some embodiments, the modified X family DNA polymerase can have less than 400 amino acids and at least about 90% or at least about 95% sequence identity to SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, or SEQ ID NO:39. In particular iterations, the modified X family DNA polymerase can consist of SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, or SEQ ID NO:39.

In certain other embodiments, the modified X family DNA polymerase can comprise a fragment of an X family DNA polymerase. For example, the modified X family DNA polymerase can have at least about 80%, 82%, 84%, 86%, 88%, 90%, 91%, 92%, 93%, 94%, 95%, 96%, 97%, 98%, or 99% sequence identity to SEQ ID NO:25 or SEQ ID NO:26. In some iterations, the modified X family DNA polymerase can consist of SEQ ID NO:25 or SEQ ID NO:26. In other embodiments, the modified X family DNA polymerase can have at least about 80%, 82%, 84%, 86%, 88%, 90%, 91%, 92%, 93%, 94%, 95%, 96%, 97%, 98%, or 99% sequence identity to SEQ ID NO:9, SEQ ID NO:11, or SEQ ID NO:13. In certain iterations, the modified X family DNA polymerase can have less than 400 amino acids and at least about 90% or at least about 95% sequence identity to SEQ ID NO:9, SEQ ID NO:11, or SEQ ID NO:13. In particular iterations, the modified X family DNA polymerase can consist of SEQ ID NO:9, SEQ ID NO:11, or SEQ ID NO:13.

### (II) Methods for Preparing Modified X Family DNA Polymerases

Another aspect of the present disclosure encompasses methods for preparing the modified X family DNA polymerases described above in section (I). In general, the methods comprise deleting, inserting, or changing one or more amino acid residues in the X family DNA polymerase, and assaying the activity of the modified X family DNA polymerase to determine if it is able to accommodate 3′-O-blocked nucleotides and synthesize polynucleotides in a template-independent manner.

Amino acid residues targeted for modification can be identified using multiple sequence alignments in which sequence similarities and differences in relevant motifs can be discerned (see FIG. 1) and/or with protein three-dimensional (3D) structure predicting programs that can identify residues that form the active site or nucleotide binding site and may interact with the bound nucleotide. Computer models also can be used to predict the fit of nucleotides comprising various 3′-O-blocking groups.

Libraries of modified X family DNA polymerase can be generated using synthesized genes, PCR site-directed mutagenesis, oligonucleotide-directed mutagenesis, saturation mutagenesis, or other techniques well known in the art.

The synthetically produced polymerase X mutant gene libraries can be expressed as recombinant proteins in one of the commonly used recombinant expression organism, E. coli, P. pastoris, as well as other eukaryotic systems. The proteins can be expressed with one or many of the affinity tags described above as to allow for an automated process of purifying the library of proteins.

Once produced in a purified and active form, the libraries of modified X family DNA polymerases can be assayed. The assay can include natural occurring dNTPs, modified blocked dNTPs, or a mixture of both in order to quantitate the activity. In some embodiments, activity can be determined by migration of a polynucleotide on a denaturing acrylamide or agarose gel. For example, gel shift assays can be used to screen the modified protein space of X family DNA polymerase variants to verify addition of 3′-O-blocked nucleotide triphosphates. In other embodiments, activity can be determined by modified fluorescent nucleotide which allows for the addition of a single blocked nucleotide that can be monitored by the excitation of the fluorescent moiety. In still other embodiments, activity can be determined by a specific increase in mass of the polynucleotide when subjected to mass spectrometry. In yet alternate embodiments, activity can be determined by Sanger sequencing to determine precise nucleotide additions. The modified X family DNA polymerases with the highest activity can be tested via an evaluation of combinatorial mutants through the same set of assays described above.

### (III) Polynucleotide Synthesis Methods

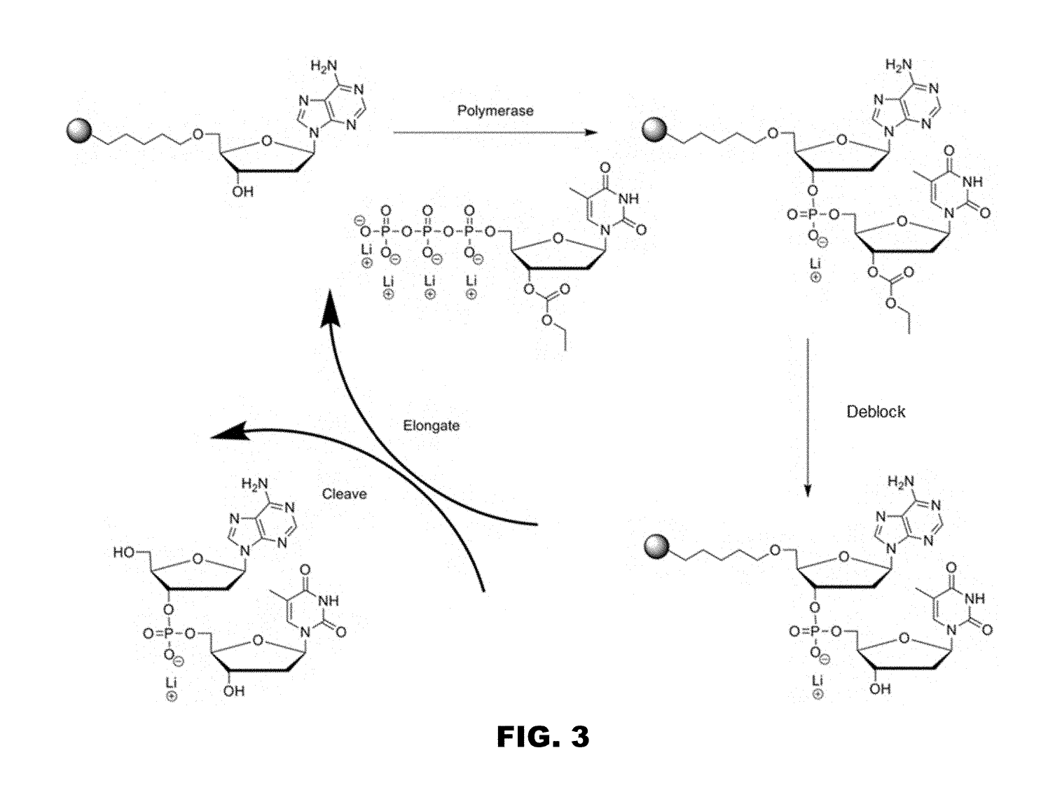

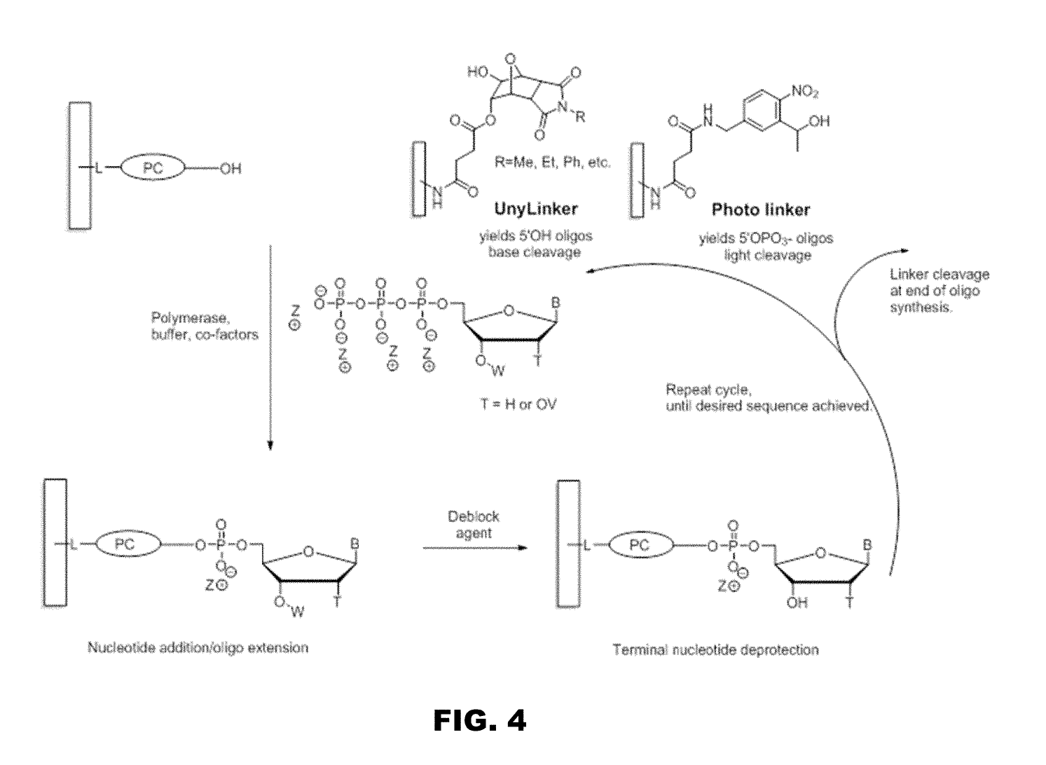

A further aspect of the present disclosure provides methods for template-independent polynucleotide synthesis using a modified X family DNA polymerase and 3′-O-blocked nucleotide 5′-triphosphates. The polynucleotide synthesis methods comprise steps of linking a 3′-O-reversibly blocked nucleotide to a free hydroxyl group to form an oligo/polynucleotide comprising a removable 3′-O-blocking group, removing the removable 3′-O-blocking group by contact with a deblocking agent to generate a free 3′-OH group, and repeating the linking and deblocking steps until the polynucleotide of the desired sequence is generated. FIGS. 3 and 4 present reaction scheme depicting polynucleotide synthesis processes.

(a) Reactants

The template-independent polynucleotide synthesis method commences with formation of a reaction phase comprising a modified X family DNA polymerase, a nucleotide 5′-triphosphase comprising a 3′-O-blocking group, and an entity comprising a free hydroxyl group.

(i) Modified X Family DNA Polymerase

The reaction phase comprises a modified X family DNA polymerase as described above in section (I). In particular, the modified X family DNA polymerase has been engineered to synthesize a single-stranded polynucleotide using 3′-O-blocked nucleotide 5′-triphosphates in the absence of a nucleic acid template.

(ii) 3-O-Reversibly Blocked Nucleotide 5′-Triphosphates.

The reaction phase also comprises a nucleotide 5′-triphosphate comprising a removable 3′-O-blocking group. A nucleotide comprises a nitrogenous base, a sugar moiety (i.e., ribose, 2′-deoxyribose, or 2′-4′ locked deoxyribose), and one or more phosphate groups. The removable 3′-O-blocking group can be an ester, ether, carbonitrile, phosphate, carbonate, carbamate, hydroxylamine, borate, nitrate, sugar, phosphoramide, phosphoramidate, phenylsulfonate, sulfate, sulfone, or amino acid.

The nucleotide 5′-triphosphate comprising the removable 3′-O-blocking group can be a deoxyribonucleotide, a ribonucleotide, or a locked nucleic acid (LNA), respectively, as diagrammed below:

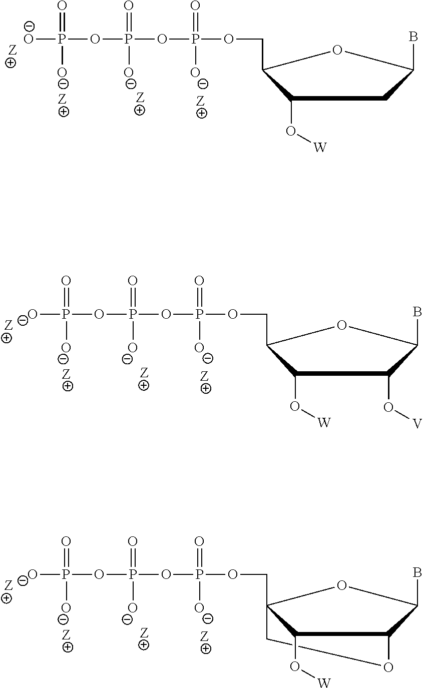

wherein:

B is a nitrogenous base;

W is a removable blocking group chosen from (CO)R, (CO)OR, (CO)CH₂OR, (CO)NHR, (CO)CH₂NHR, (CO)SR, CH₂OR, CH₂N₃, CH₂CH═CH₂, CH₂CN, NH₂, NH₃X—, NR₃X—, NHR, NRR¹, NO₂, BO₃, SOR, SO₂R, SO₃R, PO₃X₂, SiRR¹R², 2-furanyl, 2-thiofuranyl, 3-pyranyl, or 2-thiopyranylo, wherein R, R¹, and R² independently are alkyl, alkenyl, aryl, substituted alkyl, substituted alkenyl, or substituted aryl, and X is an anion;

V is hydrogen, SiRR¹R2, or CH₂OSiRR¹R², wherein R, R¹, and R² independently are alkyl, alkenyl, aryl, substituted alkyl, substituted alkenyl, or substituted aryl; and

Z is a cation.

In various embodiments, B can be a standard nucleobase, a non-standard base, a modified base, an artificial (or unnatural) base, or analog thereof. Standard nucleobases include adenine, guanine, thymine, uracil, and cytosine. In other embodiments, B can be 2-methoxy-3-methylnapthlene (NaM), 2,6-dimethyl-2H-isoquinoline-1-thione (5SICS), 8-oxo guanine (8-oxoG), 8-oxo adenine (8-oxoA), 5-methylcytosine (5mC), 5-hydroxymethyl cytosine (5hmC), 5-formyl cytosine (5fC), 5-carboxy cytosine (5caC), xanthine, hypoxanthine, 2-aminoadenine, 6-methyl or 6-alkyl adenine, 6-methyl or 6-alkyl guanine, 2-propyl or 2-alkyl adenine, 2-propyl or 2-alkyl guanine, 2-thiouracil, 2-thiothymine, 2-thiocytosine, 5-propynyl uracil, 5-propynyl cytosine, 6-azo uracil, 6-azo cytosine, 6-azo thymine, 5-uracil (pseudouracil), 4-thiouracil, 8-halo (e.g., 8-bromo) adenine, 8-amino adenine, 8-thiol adenine, 8-thioalkyl adenine, 8-hydroxyl adenine, 8-halo (e.g., 8-bromo) guanine, 8-amino guanine, 8-thiol guanine, 8-thioalkyl guanine, 8-hydroxyl guanine, 5-halo (e.g., 5-bromo) uracil, 5-trifluoromethyl uracil, 5-halo (e.g., 5-bromo) cytosine, 5-trifluoromethyl cytosine, 7-methylguanine, 7-methyladenine, 8-azaguanine, 8-azaadenine, deazaguanine, 7-deazaguanine, 3-deazaguanine, deazaadenine, 7-deazaadenine, 3-deazaadenine, pyrazolo[3,4-d]pyrimidine, inosine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine, 1,3,5 triazine, FEMO, MMO2, or TPT3.

In general, Z can be an alkali metal, an alkaline earth metal, a transition metal, NH₄, or NR₄, wherein R is alkyl, aryl, substituted alkyl, or substituted aryl. Suitable metals include sodium, potassium, lithium, cesium, magnesium, calcium, manganese, cobalt, copper, zinc, iron, and silver. In specific embodiments, Z can be lithium or sodium.

In certain embodiments, W can be (CO)R, (CO)OR, or (CO)CH₂OR, wherein R is alkyl or alkenyl. For example, W can be (CO)—O-methyl, (CO)—O-ethyl, (CO)—O-n-propyl, (CO)—O-isopropyl, (CO)—O-propenyl, (CO)—O-n-butyl, (CO)—O-t-butyl, (CO)CH₂O-methyl, (CO)CH₂O-ethyl, (CO)CH₂O-n-propyl, (CO)CH₂O-isopropyl, (CO) CH₂O-n-butyl, (CO) CH₂O-t-butyl, (CO)methyl, (CO)ethyl, (CO)n-propyl, (CO)isopropyl, (CO)n-butyl, or (CO)t-butyl. In specific embodiments, W can be (CO)—O-methyl, (CO)—O-ethyl, (CO)ethyl, (CO)n-propyl, (CO)CH₂O-methyl, or (CO)CH₂O-ethyl.

In certain embodiments, the 3′-O-reversibly blocked nucleotide 5′-triphosphate can further comprise a detectable label. The detectable label can be a detection tag such as biotin, digoxigenin, or dinitrophenyl, or a fluorescent dye such as fluorescein or derivatives thereof (e.g., FAM, HEX, TET, TRITC), rhodamine or derivatives thereof (e.g., ROX), Texas Red, cyanine dyes (e.g., Cy2, Cy3, Cy5), Alexa dyes, diethylaminocoumarin, and the like. In some embodiments, the detectable label can comprise a fluorescent dye-quencher pair. Non-limiting examples of suitable quenchers include black hole quenchers (e.g., BHQ-1, BHQ-3), Iowa quenchers, deep dark quenchers, eclipse quenchers, and dabcyl. The detectable label can be attached directly to the nitrogenous base or can be attached via a chemical linker. Suitable chemical linkers include tetra-ethylene glycol (TEG) spacers, polyethylene glycol (PEG) spacers, C6 linkers, and other linkers known in the art.

(iii) Entity with Free OH Group

The reaction phase also comprises an entity comprising a free OH group. In some embodiments, the free OH group can be a free 3′-OH group provided by a nucleotide, oligonucleotide, or polynucleotide. For example, the free OH group can be a free 3′-OH group located at the 3′ end of primer or initiator sequence. The nucleotide, oligonucleotide, or polynucleotide comprising the free 3′-OH group can be immobilized on a solid support.

In other embodiments, the entity comprising free OH group can be a solid support in which the free hydroxyl group is part of a cleavable group that is attached to the solid support. For example, the cleavable group (PC) can be linked to the solid support via a linker (L), as diagrammed below:

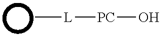

A variety of cleavable groups are suitable for linking to the solid support. The cleavable group can be cleaved by any of several mechanisms. For example, the cleavage group can be acid cleavable, base cleavable, photocleavable, electophilically cleavable, nucleophilically cleavable, cleavable under reduction conditions, cleavable under oxidative conditions, or cleavable by elimination mechanisms. Those skilled in the art are familiar with suitable cleavage sites, such as, e.g., ester linkages, amide linkages, silicon-oxygen bonds, trityl groups, tert-butyloxycarbonyl groups, acetal groups, p-alkoxybenzyl ester groups, and the like.

In specific embodiments, the cleavable group can be a photocleavable group, wherein cleavage is activated by light of a particular wavelength. Non-limiting examples of suitable photocleavable groups include nitrobenzyl, nitrophenethyl, benzoin, nitroveratryl, phenacyl, pivaloyl, sisyl, 2-hydroxy-cinamyl, coumarin-4-yl-methyl groups or derivatives thereof. In particular embodiments, the photocleavable group can be a member of the ortho-nitrobenzyl alcohol family and attached to linker L as diagrammed below.

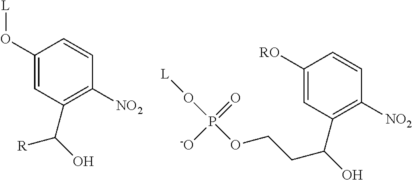

In other embodiments, the cleavable group can be a base hydrolysable group attached to linker L, as diagrammed below, wherein R can be alkyl, aryl, etc.

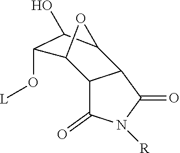

The linker (L) can be any bifunctional molecule comprising from about 6 to about 100 contiguous covalent bond lengths. For example, the linker can be an amino acid, a peptide, a nucleotide, a polynucleotide (e.g., poly A₃₋₂₀), an abasic sugar-phosphate backbone, a polymer (e.g., PEG, PLA, cellulose, and the like), a hydrocarbyl group (e.g., alkyl, alkenyl, alkynyl, aryl, aralkyl, aralkenyl, aralkynyl, and so forth), a substituted hydrocarbyl group (e.g., alkoxy, heteroaryl, aryloxy, and the like), or a combination thereof.

Specific solid supports in which the free hydroxyl group is part of a photocleavable group that is attached to the solid support via a linker (L) are diagrammed below.

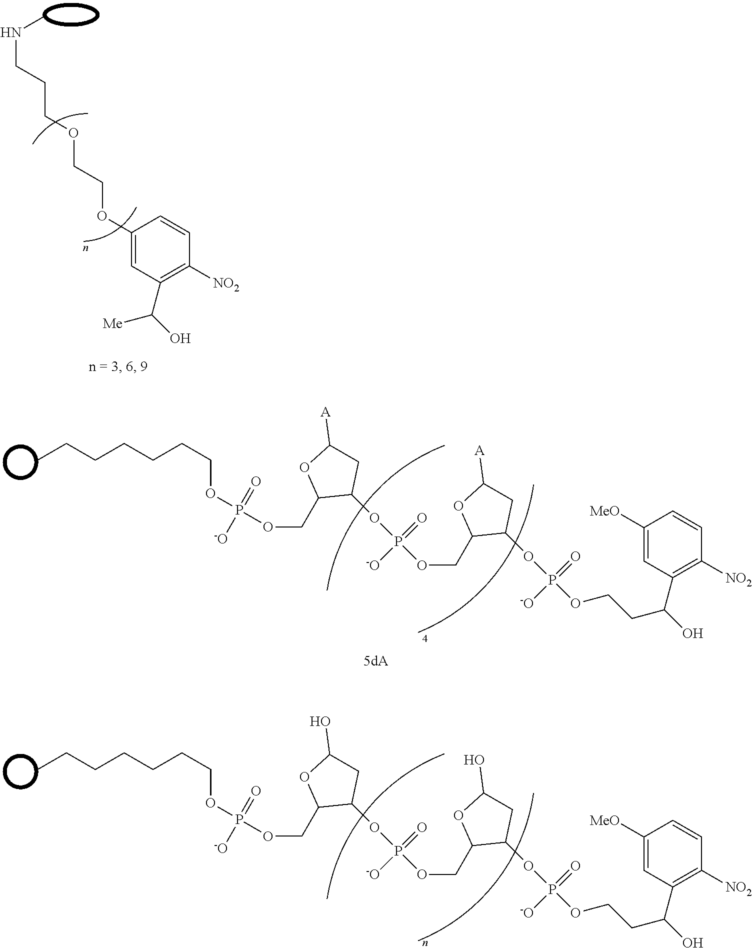

In various embodiments, the solid support can be a bead, a well, a plate, a chip, a microplate, an assay plate, a testing plate, a slide, a microtube, or any other suitable surface. The solid support can comprise polymer, plastic, resin, silica, glass, silicon, metal, carbon, or other suitable material. In certain embodiments, the solid support can be a polymer. Non-limiting examples of suitable polymers include polypropylene, polyethylene, cyclo-olefin polymer (COP), cyclo-olefin copolymer (COC), polystyrene, and polystyrene crosslinked with divinylbenzene. In specific embodiments, the polymer can be polypropylene, cyclo-olefin polymer, or cyclo-olefin copolymer.

(b) Steps of the Process

The template-independent polynucleotide synthesis method comprises cycles of linking a 3′-O-reversibly blocked nucleotide and removing the reversible 3′-O-blocking group so that another 3′-O-reversibly blocked nucleotide can be linked to the elongating polynucleotide.

(i) Linking 3′-O-Reversibly Blocked Nucleotides

The template-independent polynucleotide synthesis method disclosed herein comprises a linking step in which a nucleotide comprising a removable 3′O-blocking group is linked to a free OH group. The linking step comprises reacting the free OH group with a nucleotide 5′-triphosphate comprising a removable 3′-O-blocking group in the presence of a modified X family DNA polymerase and in the absence of a nucleic acid template. The X family DNA polymerase links the alpha 5′-phosphate group of the 3′-O-blocked nucleotide to the free OH group via a phosphodiester bond. The 3′-O-blocking group of the newly linked nucleotide prevents the addition of additional nucleotides to the oligo/polynucleotide.

The linking step generally is conducted in the presence of an aqueous solution. The aqueous solution can comprise one or more buffers (e.g., Tris, HEPES, MOPS, Tricine, cacodylate, barbital, citrate, glycine, phosphate, acetate, and the like) and one or more monovalent and/or divalent cations (e.g., Mg²⁺, Mn²⁺, Co²⁺, Cu²⁺, Zn²⁺, Na⁺, K⁺, etc. along with an appropriate counterion, such as, e.g., Cl⁻). In some embodiments, the aqueous solution can further comprise one or more nonionic detergents (e.g., Triton X-100, Tween-20, and so forth). In other embodiments, the aqueous solution can further comprise an inorganic pyrophosphatase (to counter the levels of pyrophosphate due to nucleotide triphosphate hydrolysis). The inorganic pyrophosphatase can be of yeast or bacterial (e.g., E. coli) origin. The aqueous solution generally has a pH raging from about 5 to about 10. In certain embodiments, the pH of the aqueous solution can range from about 6 to about 9, from about 6 to about 7, from about 7 to about 8, or from about 7 to about 9.

The linking step can be conducted at a temperature ranging from about 4° C. to about 80° C. In various embodiments, the temperature can range from about 4° C. to about 20° C., from about 20° C. to about 40° C., from about 40° C. to about 60° C., or from about 60° C. to about 80° C. In specific embodiments, the temperature of the linking step can range from about 20° C. to about 50° C., or from about 25° C. to about 40° C.

During the linking step, the nucleotide 5′-triphosphate comprising the removable 3′-O-blocking group can be present at a concentration ranging from about 1 μM to about 1 M. In certain embodiments, the concentration of the nucleotide 5′-triphosphate comprising a removable 3′-O-blocking group can range from about 1 μM to about to about 10 μM, from about 10 μM to about 100 μM, or from about 100 μM to about 1000 μM. The weight ratio of the solid support comprising the free hydroxyl group to the nucleotide 5′-triphosphate comprising the removable 3′-O-blocking group can range from about 1:100 to about 1:10,000. In specific embodiments, the weight ratio of the solid support comprising the free hydroxyl group to the nucleotide 5′-triphosphate comprising the removable 3′-O-blocking group can range from about 1:500 to about 1:2000.

In general, the amount of the X family DNA polymerase present during the linking step will be sufficient to catalyze the reaction in a reasonable period of time. In general, the linking step is allowed to proceed until the phosphodiester bond formation is complete. The formation of the phosphodiester bond can be monitored by incorporating a 3′-O-blocked nucleotide comprising a fluorescent label.

At the end of the linking step, the X family DNA polymerase and the unreacted 3′-O-reversibly blocked nucleotide 5′-triphosphate generally are removed from the immobilized nucleotide. In some embodiments, the aqueous solution comprising the X family DNA polymerase and the unreacted 3′-O-reversibly blocked nucleotide 5′-triphosphate can be removed, optionally recycled, and replaced with aqueous solution (e.g., fresh or recycled aqueous solution that is used during the deblocking step, described below). In other embodiments, the X family DNA polymerase can be removed from the aqueous solution by contact with an antibody that recognizes the X family DNA polymerase. In still other embodiments, the aqueous solution comprising the X family DNA polymerase and/or the unreacted 3′-O-reversibly blocked nucleotide 5′-triphosphate can be washed or flushed away with a wash solution. The wash solution can comprise the same components as used during the deblocking step.

(ii) Removing the 3′-O-Removable Blocking Group

The method further comprises a deblocking step in which the removable 3′-O-blocking group is removed from the 3′-O-blocked nucleotide linked to the oligo/polynucleotide. The deblocking step comprises contacting the linked nucleotide comprising the removable 3′-O-blocking group with a deblocking agent, thereby removing the 3′-O-blocking group and creating a free hydroxyl group on the oligo/polynucleotide.

The type and amount of deblocking agent will depend upon the identity of the removable 3′-O-blocking group. Suitable deblocking agents include acids, bases, nucleophiles, electrophiles, radicals, metals, reducing agents, oxidizing agents, enzymes, and light. In embodiments in which the blocking group comprises an ester or carbamate linkage, the deblocking agent can be a base (e.g., an alkali metal hydroxide). In instances in which the blocking group comprises an ether linkage, the deblocking agent can be an acid. In embodiments in which when the blocking group is O-amino, the deblocking agent can be sodium nitrite. In aspects in which the blocking group is O-allyl, the deblocking agent can be a transition metal catalyst. In embodiments in which the blocking group is azidomethyl, the deblocking agent can be a phosphine (e.g., tris(2-carboxyethyl)phosphine). In embodiments in which the blocking group comprises an ester or carbonate linkage, the deblocking agent can be an esterase or lipase enzyme. The esterase or lipase enzyme can be derived from animal, plant, fungi, archaeal, or bacterial sources. The esterase or lipase can be mesophilic or thermophilic. In one embodiment, the esterase can be derived from porcine liver.

In general, the deblocking step is conducted in the presence of an aqueous solution. That is, the deblocking agent can be provided as an aqueous solution comprising the deblocking agent. In some embodiments, the aqueous solution can comprise one or more protic, polar solvents. Suitable protic, polar solvents include water; alcohols such as methanol, ethanol, isopropanol, n-propanol, isobutanol, n-butanol, s-butanol, t-butanol, and the like; diols such as glycerol, propylene glycol and so forth; organic acids such as formic acid, acetic acid, and so forth; an amine such as triethylamine, morpholine, piperidine, and the like; and combinations of any of the above. In other embodiments, the aqueous solution can comprise one or more buffers (e.g., Tris, HEPES, MOPS, Tricine, cacodylate, barbital, citrate, glycine, phosphate, acetate, and the like). In still other embodiments, the aqueous solution can further comprise one or more denaturants to disrupt any secondary structures in the oligo/polynucleotides. Suitable denaturants include urea, guanidinium chloride, formamide, and betaine.

The pH of the aqueous solution can range from about 1 to about 14, depending upon the identity of the deblocking agent. In various embodiments, the pH of the aqueous solution can range from about 2 to about 13, from about 3 to about 12, from about 4 to about 11, from 5 to about 10, from about 6 to about 9, or from about 7 to about 8. In specific embodiments, the pH of the aqueous solution comprising the deblocking agent can range from about 10 to about 14, or from about 11 to about 13.

In embodiments in which the deblocking agent is an esterase or lipase enzyme, the enzyme can be provided in a buffered aqueous solution having a pH from about 6.5 to about 8.5.

The deblocking step can be performed at a temperature ranging from about 0° C. to about 100° C. In some embodiments, the temperature can range from about 4° C. to about 90° C. In various embodiments, the temperature can range from about 0° C. to about 20° C., from about 20° C. to about 40° C., from about 40° C. to about 60° C., from about 60° C. to about 80° C., or from about 80° C. to about 100° C. In certain embodiments, then deblocking step can be performed at about 60° C. to about 80° C. The deblocking step can be performed at a first temperature, followed by a second temperature. For example, the aqueous solution comprising the deblocking agent can be provided at one temperature and then the temperature can be raised to assist in cleavage and disrupt any secondary structure.

The duration of the deblocking step will vary depending upon the nature of the protecting chemistry and type of deblocking agent. In general, the deblocking step is allowed to proceed until the reaction has gone to completion, as determined by methods known in the art.

At the end of the deblocking step, the deblocking agent generally is removed from the immobilized nucleotide bearing the free hydroxyl group. In some embodiments, the aqueous solution comprising the deblocking agent can be removed, optionally recycled, and replaced with aqueous solution (e.g., fresh or recycled aqueous solution that is used during the linking step, as described above). In other embodiments, the aqueous solution comprising the deblocking agent can be washed or flushed away with a wash solution. The wash solution can comprise the same buffers and salts as used during the linking step. In embodiments in which the deblocking agent is an enzyme, the enzyme can be removed from the aqueous solution by contact with an antibody that recognizes the enzyme.

In specific embodiments, the removable 3′-O-blocking group is linked to the nucleotide 5′-triphosphase via an ester or carbonate linkage, and the deblocking agent is a base or an esterase or lipase enzyme.

(iii) Repeating the Linking and Deblocking Steps

The steps of linking a 3′-O-blocked nucleotide and removing the removable blocking group can be repeated until the polynucleotide of the desired length and sequence is achieved.

The linking and deblocking steps can be performed in a microfluidic instrument, a column-based flow instrument, or an acoustic droplet ejection (ADE)-based system. The aqueous solution comprising the appropriate 3′-O-blocked nucleotide 5′-triphosphate and the modified X family DNA polymerase, the aqueous solution comprising the deblocking agent, wash solutions, etc., can be dispensed through acoustic transducers or microdispensing nozzles using any applicable jetting technology, including piezo or thermal jets. The temperature and duration of each step can be controlled by a processing unit.

In embodiments in which the newly synthesized polynucleotide is immobilized on a solid support, the method can further comprise releasing the polynucleotide using methods known in the art.

(iv) Synthesized Polynucleotide

In embodiments in which the newly synthesized polynucleotide is immobilized on a solid support, the polynucleotide can be released by methods known in the art. For example, if the polynucleotide is linked to a solid support via a photocleavable group linker, the photocleavable linker can be cleaved by contact with light of a suitable wavelength.

The polynucleotides synthesized by the methods described herein can be deoxyribonucleic acid (DNA), ribonucleic acid (RNA), locked nucleic acid (LNA), or a combination thereof. In general, the polynucleotides prepared by the methods disclosed herein are single stranded. In embodiments in which the polynucleotide is DNA, the single-stranded DNA can be converted to double-stranded DNA by contact with a DNA polymerase (as well as suitable primers and dNTPs). The DNA polymerase can be thermophilic or mesophilic. Suitable DNA polymerases include Taq DNA polymerase, Pfu DNA polymerase, Pfx DNA polymerase, Tli (also known as Vent) DNA polymerase, Tfl DNA polymerase, Tth DNA polymerase, Tko DNA polymerase (also known as KOD), E. coli DNA polymerase I, T4 DNA polymerase, T7 DNA polymerase, variants thereof, and engineered versions thereof.

The lengths of polynucleotides synthesized by the methods described herein can range from about several nucleotides (nt) to hundreds of thousands or millions of nt. In various embodiments, the polynucleotide can comprise from about 4 nt to about 30 nt, from about 30 nt to about 100 nt, from about 100 nt to about 300 nt, from about 300 nt to about 1000 nt, from about 1000 nt to about 3000 nt, from about 3,000 nt to about 10,000, from about 10,000 nt to about 100,000 nt, from about 100,000 nt to about 1,000,000 nt, or from about 1,000,000 nt to about 10,000,000 nt.

As such, the methods disclosed herein can be used to synthesize whole genes or synthetic genes for research, clinical, diagnostic, and/or therapeutic applications. Similar, the methods disclosed herein can be used to synthesize whole plasmids, synthetic plasmids, and/or synthetic viruses (e.g., DNA or RNA) for a variety of applications. Additionally, the methods disclosed herein can be used to synthesize long synthetic RNAs for a variety of research and/or diagnostic/therapeutic applications.

### Enumerated Embodiments

The following enumerated embodiments are presented to illustrate certain aspects of the present invention, and are not intended to limit its scope.

1. A modified X family DNA polymerase comprising SEQ ID NO:1 inserted into a loop 1 region, wherein the modified X family DNA polymerase is other than a terminal deoxynucleotidyl transferase or human DNA polymerase mu.

2. The modified X family DNA polymerase of embodiment 1, wherein the modified X family DNA polymerase is capable of accommodating a nucleotide 5′-triphosphate comprising a removable 3′-O-blocking group.

3. The modified X family DNA polymerase of embodiments 1 or 2, wherein the removable 3′-O-blocking group is chosen from (CO)R, (CO)OR, (CO)CH₂OR, (CO)NHR, (CO)CH₂NHR, (CO)SR, CH₂OR, CH₂N₃, CH₂CH═CH₂, CH₂CN, or NH₂, wherein R is alkyl or alkenyl.

4. The modified X family DNA polymerase of any one of embodiments 1 to 3, wherein the modified X family DNA polymerase is capable of adding a 3′-O-blocked nucleotide to a free hydroxyl group in the absence of a nucleic acid template.

5. The modified X family DNA polymerase of any one of embodiments 1 to 4, wherein the modified X family DNA polymerase is chosen from (i) a polypeptide of less than about 400 amino acids that has at least about 90% sequence identity to SEQ ID NO:16, 27, 28, 29, 30, 31, 32, 33, 34, 35, 36, 37, 38, or 39; or (ii) a polypeptide having at least about 90% sequence identity to SEQ ID NO:18, 19, 21, or 23.

6. The modified X family DNA polymerase of any one of embodiments 1 to 5, wherein (i) has at least about 95% sequence identity to SEQ ID NO:16, 27, 28, 29, 30, 31, 32, 33, 34, 35, 36, 37, 38, or 39.

7. The modified X family DNA polymerase of embodiment 6, wherein (i) consists of SEQ ID NO:16, 27, 28, 29, 30, 31, 32, 33, 34, 35, 36, 37, 38, or 39.

8. The modified X family DNA polymerase of any one of embodiments 1 to 5, wherein (ii) has at least about 95% sequence identity to SEQ ID NO: 18, 19, 21, or 23.

9. The modified X family DNA polymerase of embodiment 8, wherein (ii) consists of SEQ ID NO:18, 19, 21, or 23.

10. The modified X family DNA polymerase of any one of embodiments 1 to 9, wherein the modified X family DNA polymerase further comprises at least one marker domain, at least one purification tag, or combination thereof at the N-terminal end, the C-terminal end, or both.

11. A method for synthesizing a polynucleotide comprising (a) providing an entity comprising a free hydroxyl group; (b) contacting the free hydroxyl group with a nucleotide 5′-triphosphate comprising a removable 3′-O-blocking group in the presence of a modified X family DNA and in the absence of a nucleic acid template to form a linked nucleotide comprising a removable 3′-O-blocking group, wherein the modified X family DNA polymerase comprises SEQ ID NO:1 inserted into a loop 1 region and is other than a terminal deoxynucleotidyl transferase; (c) contacting the linked nucleotide comprising the removable 3′-O-blocking group with a deblocking agent to remove the removable 3′-O-blocking group; and (d) repeating steps (b) and (c) to yield the polynucleotide.

12. The method of embodiment 11, wherein the free hydroxyl group is a free 3′OH group of an initiator sequence, an oligonucleotide, or a polynucleotide.

13. The method of embodiment 11, wherein the free hydroxyl group is part of a cleavable group attached to a solid support by a linker.

14. The method of any one of embodiments 11 to 13, wherein the nucleotide 5′-triphosphate comprising the removable 3′-O-blocking group has a sugar moiety chosen from ribose, 2′-deoxyribose, or 2′-4′ locked deoxyribose and a nitrogenous base chosen from a standard nucleobase, a non-standard base, a modified base, an artificial base, or an analog thereof.

15. The method of any one of embodiments 11 to 14, wherein the removable 3′-O-blocking group is chosen from (CO)R, (CO)OR, (CO)CH₂OR, (CO)NHR, (CO)CH₂NHR, (CO)SR, CH₂OR, CH₂N₃, CH₂CH═CH₂, CH₂CN, or NH₂, wherein R is alkyl or alkenyl.

16. The method of any one of embodiments 11 to 15, wherein the removable 3′-O-blocking group is chosen from (CO)—O-methyl, (CO)—O-ethyl, (CO)—O-n-propyl, (CO)—O-isopropyl, (CO)—O-propenyl, (CO)—O-n-butyl, (CO)—O-t-butyl, (CO)CH₂O-methyl, (CO)CH₂O-ethyl, (CO)CH₂O-n-propyl, (CO)CH₂O-isopropyl, (CO) CH₂O-n-butyl, (CO) CH₂O-t-butyl, (CO)methyl, (CO)ethyl, (CO)n-propyl, (CO)isopropyl, (CO)n-butyl, or (CO)t-butyl.

17. The method of any one of embodiments 11 to 16, wherein the modified X family DNA polymerase has at least about 90% sequence identity to SEQ ID NO:15, 16, 18, 19, 21, 23, 27, 28, 29, 30, 31, 32, 33, 34, 35, 36, 37, 38, or 39.

18. The method of any one of embodiments 11 to 17, wherein the modified X family DNA polymerase consists of SEQ ID NO:15, 16, 18, 19, 21, 23, 27, 28, 29, 30, 31, 32, 33, 34, 35, 36, 37, 38, or 39.

19. The method of any one of embodiments 11 to 18, wherein the deblocking agent at step (c) is an acid, a base, a nucleophile, an electrophile, a radical, a metal, a reducing agent, an oxidizing agent, an enzyme, or light.

20. The method of any one of embodiments 11 to 19, wherein the deblocking agent at step (c) is a base or an esterase or lipase enzyme.

21. The method of any one of embodiments 11 to 20, wherein the entity comprising the free hydroxyl group and the nucleotide 5′-triphosphate comprising the removable 3′-O-blocking group are present at a weight ratio from about 1:500 to about 1:2000.

22. The method of any one of embodiments 11 to 21, wherein step (b) is performed at a temperature from about 20° C. to about 50° C. in the presence of an aqueous solution having a pH from about 7 to 9.

23. The method of any one of embodiments 11 to 22, wherein the modified X family DNA polymerase and unreacted nucleotide 5′-triphosphate comprising the removable 3′-O-blocking group are removed at the end of step (b) and optionally recycled.

24. The method of any one of embodiments 11 to 22, wherein the modified X family DNA polymerase is removed at the end of step (b) by contact with an antibody that recognizes the modified X family DNA polymerase.

25. The method of any one of embodiments 11 to 24, wherein step (b) is followed by a washing step to remove the modified X family DNA polymerase and unreacted nucleotide 5′-triphosphate comprising the removable 3′-O-blocking group.

26. The method of any one of embodiments 11 to 25, wherein step (c) is performed at a temperature from about 4° C. to about 90° C.

27. The method of any one of embodiments 11 to 26, wherein the deblocking agent is removed at the end of step (c) and optionally recycled.

28. The method of any one of embodiments 11 to 27, wherein step (c) is followed by a washing step to remove the deblocking agent.

29. The method of any one of embodiments 11 to 28, where the polynucleotide is DNA, RNA, locked nucleic acid (LNA), or a combination thereof, and has a length from about ten nucleotides to hundreds of thousands of nucleotides.

### Definitions

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

The term “alkyl” as used herein describes saturated hydrocarbyl groups that contain from 1 to 30 carbon atoms. They may be linear, branched, or cyclic, may be substituted as defined below, and include methyl, ethyl, propyl, isopropyl, butyl, hexyl, heptyl, octyl, nonyl, and the like.

The term “alkenyl” as used herein describes hydrocarbyl groups which contain at least one carbon-carbon double bond and contain from 1 to 30 carbon atoms. They may be linear, branched, or cyclic, may be substituted as defined below, and include ethenyl, propenyl, isopropenyl, butenyl, isobutenyl, hexenyl, and the like.

The term “alkoxy” as used herein is the conjugate base of an alcohol. The alcohol may be straight chain, branched, or cyclic.

The term “alkynyl” as used herein describes hydrocarbyl groups which contain at least one carbon-carbon triple bond and contain from 1 to 30 carbon atoms. They may be linear or branched, may be substituted as defined below, and include ethynyl, propynyl, butynyl, isobutynyl, hexynyl, and the like.

The term “aryl” as used herein alone or as part of another group denote optionally substituted homocyclic aromatic groups, preferably monocyclic or bicyclic groups containing from 6 to 10 carbons in the ring portion, such as phenyl, biphenyl, naphthyl, substituted phenyl, substituted biphenyl, or substituted naphthyl.

The terms “halogen” or “halo” as used herein alone or as part of another group refer to chlorine, bromine, fluorine, and iodine.

The term “heteroatom” refers to atoms other than carbon and hydrogen.

The term “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. They may be straight, branched, or cyclic. Unless otherwise indicated, these moieties preferably comprise 1 to 20 carbon atoms.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “substituted hydrocarbyl, “substituted alkyl,” “substituted aryl,” and the like refer to said moieties substituted with at least one atom other than carbon, including moieties in which a carbon chain atom is substituted with a heteroatom such as nitrogen, oxygen, silicon, phosphorous, boron, or a halogen atom, and moieties in which the carbon chain comprises additional substituents. These substituents include alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxyl, keto, ketal, phospho, nitro, and thio.

Having described the invention in detail, it will be apparent that modifications and variations are possible without departing from the scope of the invention defined in the appended claims.

### Examples

The following examples illustrate certain aspects of the disclosure.

### Example 1. Generation of Modified X Family DNA Polymerases

DNA encoding human DNA pol mu, human DNA Pol lambda, human DNA Pol beta, human DNA pol theta, ASFV DNA pol X, bovine TdT, mouse TdT, and S. harrisii TdT, or fragments thereof was generated and cloned using standard procedures. N-terminal truncations, insertions (i.e., insertion/swap of loop 1 domain of human TdT (i.e., SEQ ID NO:1)), and point mutations were prepared using standard procedures. The proteins were expressed in E. coli cells as N-terminal tagged protein and purified accordingly.

Table 1 lists the X family DNA polymerases that were generated.

TABLE 1
X Family DNA Polymerases

| Protein | Species | Description | Name | SEQ ID NO |
|---|---|---|---|---|
| TdT | Bos taurus | Wild type | Bt TdT | 8 |
| TdT | Bos taurus | N-terminal truncation (Δ1-138) | Bt tTdT | 9 |
| TdT | Mus | Wild type | Mm TdT | 10 |
| | musculus | | | |
| TdT | Mus | N-terminal truncation (Δ 1-127) | Mm tTdT | 11 |
| | musculus | | | |
| TdT | Sarciphilus | Wild type | Sh TdT | 12 |
| | harrisii | | | |
| TdT | Sarciphilus | N-terminal truncation (Δ 1-128) | Hs tTdT | 13 |
| | harrisii | | | |
| PolM | Homo | Wild type | Hs PolM | 14 |
| | sapiens | | | |
| PolM | Homo | Loop 1 domain swap | Hs PolM-Lp1 | 15 |
| | sapiens | | | |
| PolM | Homo | Loop 1 domain swap and N- | Hs tPolM-Lp1 | 16 |
| | sapiens | terminal truncation (Δ 1-108) | | |
| PolL | Homo | Wild type | Hs PolL | 17 |
| | sapiens | | | |
| PolL | Homo | Loop 1 domain swap/insertion | Hs PolL-Lp1 | 18 |
| | sapiens | | | |
| PolL | Homo | Loop 1 domain swap/insertion and | Hs tPolL-Lp1 | 19 |
| | sapiens | N-terminal truncation (Δ 1-205) | | |
| PolB | Homo | Wild type | Hs PolB | 20 |
| | sapiens | | | |
| PolB | Homo | Loop 1 domain swap/insertion | Hs PolB-Lp1 | 21 |
| | sapiens | | | |
| PolX | ASFV | Wild type | ASFV PolX | 22 |
| PolX | ASFV | Loop 1 domain swap/insertion | ASFV PolX- | 23 |
| | | | Lp1 | |
| PolQ | Homo | Wild type | Hs PolQ | 24 |
| | sapiens | | | |
| PolQ | Homo | Polymerase-like domain (PLD) (aa | Hs PolQ-PLD | 25 |
| | sapiens | 1819-2590) | | |
| PolQ | Homo | Helicase-like domain (HLD) (aa | Hs PolQ-HLD | 26 |
| | sapiens | 67-894) | | |
| PolM | Homo | Loop 1 domain swap and N- | Hs tPolM-Lp1 | 27 |
| | sapiens | terminal truncation and C284L | C284L | |
| PolM | Homo | Loop 1 domain swap and N- | Hs tPolM-Lp1 | 28 |
| | sapiens | terminal truncation and K344H | K344H | |
| PolM | Homo | Loop 1 domain swap and N- | Hs tPolM-Lp1 | 29 |
| | sapiens | terminal truncation and L184S | L184S | |
| PolM | Homo | Loop 1 domain swap and N- | Hs tPolM-Lp1 | 30 |
| | sapiens | terminal truncation and L219E and | L219E/Q220F | |
| | | Q220F | | |
| PolM | Homo | Loop 1 domain swap and N- | Hs tPolM-Lp1 | 31 |
| | sapiens | terminal truncation and L219E | L219E | |
| PolM | Homo | Loop 1 domain swap and N- | Hs tPolM-Lp1 | 32 |
| | sapiens | terminal truncation and L333Q | L333Q | |
| PolM | Homo | Loop 1 domain swap and N- | Hs tPolM-Lp1 | 33 |
| | sapiens | terminal truncation and P182C | | |
| PolM | Homo | Loop 1 domain swap and N- | Hs tPolM-Lp1 | 34 |
| | sapiens | terminal truncation and P322A | P182C | |
| PolM | Homo | Loop 1 domain swap and N- | Hs tPolM-Lp1 | 35 |
| | sapiens | terminal truncation and Q220F | Q220F/Q335E | |
| | | and Q335E | | |
| PolM | Homo | Loop 1 domain swap and N- | Hs tPolM-Lp1 | 36 |
| | sapiens | terminal truncation and Q220F | Q220F | |
| PolM | Homo | Loop 1 domain swap and N- | Hs tPolM-Lp1 | 37 |
| | sapiens | terminal truncation and Q335E | Q335E | |
| PolM | Homo | Loop 1 domain swap and N- | Hs tPolM-Lp1 | 38 |
| | sapiens | terminal truncation and R343T | R343T/K342H | |
| | | and K342H | | |
| PolM | Homo | Loop 1 domain swap and N- | Hs tPolM-Lp1 | 39 |
| | sapiens | terminal truncation and R343T | R343T | |

### Example 2. Incorporation of 3′O-Blocked Nucleotides by Modified X Family DNA Polymerase

The ability of the PolM-loop1 chimera, Hs PolM-Lp1, to incorporate 3′-O-blocked nucleotides was examined in a template-free DNA synthesis reaction. The removable blocking groups were carbamate or ester groups, as indicated in Table 2.

TABLE 2
3′-O-Carbamate or Ester dNTPs

| 3′-O-dNTP | Blocking Group |
|---|---|
| dNTP1 | —(CO)—O-methyl |
| dNTP2 | —(CO)-ethyl |
| dNTP3 | —(CO)-propyl |
| dNTP5 | —(CO)-methyl |
| dNTP6 | —(CO)—O-ethyl |

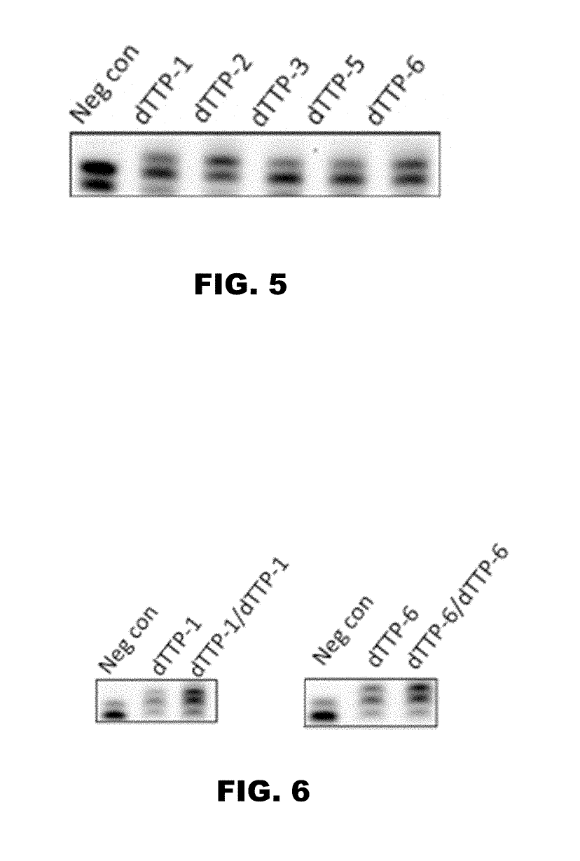

As shown in FIG. 5, Hs PolM-Lp1 successfully incorporated the 3′-O-carbamate or ester blocked nucleotides.

The carbamate or ester blocking groups were removed by contact with heat and high pH solution (e.g., pH 12 at 70° C.). Compete removal of the blocking group was confirmed by HPLC. Multiple cycles of incorporating 3′-O-carbamate or ester blocked nucleotides using Hs PolM-Lp1 followed by deblocking are presented in FIG. 6.

### Example 3. Comparison of Mutant and Wild Type X Family DNA Polymerases

The incorporation of 3′-O-carbamate or ester blocked nucleotides by the PolM-loop1 chimera, Hs PolM-Lp1, or the truncated PolM-loop1 chimera, Hs tPolM-Lp1 was compared to that of wild type Hs PolM. The amount of incorporation was quantified by densitometry. As shown in Table 3, Hs PolM-Lp1 and Hs tPolM-Lp1 showed significantly increased rates of incorporation of 3′-O-carbamate or ester blocked nucleotides as compared to wild type (WT) Hs PolM. The effect was even more dramatic with the use of a 3′-O-blocked non-natural nucleotide (d5SISC).

TABLE 3
Comparison of Mutant and Wild Type Polymerases

| | Incorporation | | Fold increase |
|---|---|---|---|
| | Hs PolM- | Incorporation Hs | Hs tPolM-Lp1 vs. |
| Blocking group | Lp1 vs. WT | tPolM-Lp1 vs. WT | Hs PolM-Lp1 |
| 1 | ++ | +++ | 2.2 |
| 2 | + | ++ | 2.1 |
| 3 | ++ | +++ | 2.0 |
| 5 | + | + | 1.3 |
| 6 (standard | + | ++ | 2.0 |
| base) | | | |
| 6 (artificial | +++++ | ++++++++++ | 3.0 |
| base-5SICS) | | +++++++ | |

TdT does not incorporate 3′-O-blocked adenosine 5′-triphosphates very efficiently. A comparison of the incorporation of 3′-O-blocked adenosine by Hs tPolM-Lp1 and Bt TdT revealed that Hs tPolM-Lp1 exhibited a 2.7 fold increase in incorporation relative to Bt TdT.

What is claimed is:

1. A modified X family DNA polymerase comprising SEQ ID NO:1 inserted into a loop 1 region, wherein the modified X family DNA polymerase is other than a terminal deoxynucleotidyl transferase or human DNA polymerase mu.

2. The modified X family DNA polymerase of claim 1, wherein the modified X family DNA polymerase is capable of accommodating a nucleotide 5′-triphosphate comprising a removable 3′-O-blocking group.

3. The modified X family DNA polymerase of claim 2, wherein the removable 3′-O-blocking group is chosen from (CO)R, (CO)OR, (CO)CH₂OR, (CO)NHR, (CO)CH₂NHR, (CO)SR, CH₂OR, CH₂N₃, CH₂CH═CH₂, CH₂CN, or NH₂, wherein R is alkyl or alkenyl.

4. The modified X family DNA polymerase of claim 1, wherein the modified X family DNA polymerase is capable of adding a 3′-O-blocked nucleotide to a free hydroxyl group in the absence of a nucleic acid template.

5. The modified X family DNA polymerase of claim 1, wherein the modified X family DNA polymerase is chosen from:
(i) a polypeptide of less than about 400 amino acids that has at least about 90% sequence identity to SEQ ID NO:16, 27, 28, 29, 30, 31, 32, 33, 34, 35, 36, 37, 38, or 39; or
(iii) a polypeptide having at least about 90% sequence identity to SEQ ID NO:18, 19, 21, or 23.

6. The modified X family DNA polymerase of claim 5, wherein (i) has at least about 95% sequence identity to SEQ ID NO:16, 27, 28, 29, 30, 31, 32, 33, 34, 35, 36, 37, 38, or 39.

7. The modified X family DNA polymerase of claim 6, wherein (i) consists of SEQ ID NO:16, 27, 28, 29, 30, 31, 32, 33, 34, 35, 36, 37, 38, or 39.

8. The modified X family DNA polymerase of claim 5, wherein (ii) has at least about 95% sequence identity to SEQ ID NO: 18, 19, 21, or 23.

9. The modified X family DNA polymerase of claim 8, wherein (ii) consists of SEQ ID NO:18, 19, 21, or 23.

10. The modified X family DNA polymerase of claim 1, wherein the modified X family DNA polymerase further comprises at least one marker domain, at least one purification tag, or combination thereof at the N-terminal end, the C-terminal end, or both.

11. A method for synthesizing a polynucleotide comprising:
(a) providing an entity comprising a free hydroxyl group;
(b) contacting the free hydroxyl group with a nucleotide 5′-triphosphate comprising a removable 3′-O-blocking group in the presence of a modified X family DNA and in the absence of a nucleic acid template to form a linked nucleotide comprising a removable 3′-O-blocking group, wherein the modified X family DNA polymerase comprises SEQ ID NO:1 inserted into a loop 1 region and is other than a terminal deoxynucleotidyl transferase;
(c) contacting the linked nucleotide comprising the removable 3′-O-blocking group with a deblocking agent to remove the removable 3′-O-blocking group; and
(d) repeating steps (b) and (c) to yield the polynucleotide.

12. The method of claim 11, wherein the free hydroxyl group is a free 3′OH group of an initiator sequence, an oligonucleotide, or a polynucleotide.

13. The method of claim 11, wherein the free hydroxyl group is part of a cleavable group attached to a solid support by a linker.

14. The method of claim 11, wherein the nucleotide 5′-triphosphate comprising the removable 3′-O-blocking group has a sugar moiety chosen from ribose, 2′-deoxyribose, or 2′-4′ locked deoxyribose and a nitrogenous base chosen from a standard nucleobase, a non-standard base, a modified base, an artificial base, or an analog thereof.

15. The method of claim 14, wherein the removable 3′-O-blocking group is chosen from (CO)R, (CO)OR, (CO)CH₂OR, (CO)NHR, (CO)CH₂NHR, (CO)SR, CH₂OR, CH₂N₃, CH₂CH═CH₂, CH₂CN, or NH₂, wherein R is alkyl or alkenyl.

16. The method of claim 15, wherein the removable 3′-O-blocking group is chosen from (CO)—O-methyl, (CO)—O-ethyl, (CO)—O-n-propyl, (CO)—O-isopropyl, (CO)—O-propenyl, (CO)—O-n-butyl, (CO)—O-t-butyl, (CO)CH₂O-methyl, (CO)CH₂O-ethyl, (CO)CH₂O-n-propyl, (CO)CH₂O-isopropyl, (CO) CH₂O-n-butyl, (CO) CH₂O-t-butyl, (CO)methyl, (CO)ethyl, (CO)n-propyl, (CO)isopropyl, (CO)n-butyl, or (CO)t-butyl.

17. The method of claim 11, wherein the modified X family DNA polymerase has at least about 90% sequence identity to SEQ ID NO: 15, 16, 18, 19, 21, 23, 27, 28, 29, 30, 31, 32, 33, 34, 35, 36, 37, 38, or 39.

18. The method of claim 11, wherein the modified X family DNA polymerase consists of SEQ ID NO:15, 16, 18, 19, 21, 23, 27, 28, 29, 30, 31, 32, 33, 34, 35, 36, 37, 38, or 39.

19. The method of claim 11, wherein the deblocking agent at step (c) is an acid, a base, a nucleophile, an electrophile, a radical, a metal, a reducing agent, an oxidizing agent, an enzyme, or light.

20. The method of claim 16, wherein the deblocking agent at step (c) is a base or an esterase or lipase enzyme.

21. The method of claim 11, wherein the entity comprising the free hydroxyl group and the nucleotide 5′-triphosphate comprising the removable 3′-O-blocking group are present at a weight ratio from about 1:500 to about 1:2000.

22. The method of claim 11, wherein step (b) is performed at a temperature from about 20° C. to about 50° C. in the presence of an aqueous solution having a pH from about 7 to 9.

23. The method of claim 11, wherein the modified X family DNA polymerase and unreacted nucleotide 5′-triphosphate comprising the removable 3′-O-blocking group are removed at the end of step (b) and optionally recycled.

24. The method of claim 11, wherein the modified X family DNA polymerase is removed at the end of step (b) by contact with an antibody that recognizes the modified X family DNA polymerase.

25. The method of claim 11, wherein step (b) is followed by a washing step to remove the modified X family DNA polymerase and unreacted nucleotide 5′-triphosphate comprising the removable 3′-O-blocking group.

26. The method of claim 11, wherein step (c) is performed at a temperature from about 4° C. to about 90° C.

27. The method of claim 11, wherein the deblocking agent is removed at the end of step (c) and optionally recycled.

28. The method of claim 11, wherein step (c) is followed by a washing step to remove the deblocking agent.

29. The method of claim 11, where the polynucleotide is DNA, RNA, locked nucleic acid (LNA), or a combination thereof, and has a length from about ten nucleotides to hundreds of thousands of nucleotides.
